(19) United States
(12) Patent Application Publication
(10) Pub. No.: US 2025/0268975 A1
(43) Pub. Date: Aug. 28, 2025

(54) PEPTOIDS FOR TREATING FILOVIRIDAE INFECTIONS

(71) Applicant: Maxwell Biosciences, Inc., Austin, TX (US)

(72) Inventors: Sheetal Vali, Dallas, TX (US); Donald J. Treacy, Woodbine, MD (US); Beth Burnside, Santa Rosa Beach, FL (US); Joshua McClure, Austin, TX (US)

(21) Appl. No.: 19/057,603

(22) Filed: Feb. 19, 2025

Related U.S. Application Data
(60) Provisional application No. 63/557,200, filed on Feb. 23, 2024.

(51) Int. Cl. A61K 38/08

(52) U.S. Cl. CPC A61K 38/08

(57) ABSTRACT

Methods of using peptoid compounds for preventing or treating an infection of a virus of the Filoviridae family are described.

### RELATED APPLICATIONS

This application claims the benefit of U.S. Provisional Application No. 63/557,200 filed Feb. 23, 2024, the contents of which is incorporated by reference herein in its entirety.

### TECHNICAL FIELD

The present disclosure relates to methods of using peptoid compounds for treating or preventing Filovirus infections in humans and animals.

### BACKGROUND

Viruses in the family Filoviridae can cause severe and deadly infections known as hemorrhagic fever in humans and nonhuman primates (such as monkeys and gorillas) and may also spread to other animals, such as bats. Filoviruses are enveloped in a lipid (fatty) membrane. The two main types of Filoviridae pathogens causing hemorrhagic fever are due to infection with the Marburg virus or the Ebola virus. Ebola Virus Disease (EVD), is a severe, often fatal illness caused by the Ebola virus, which belongs to the family Filoviridae. EVD outbreaks have occurred predominantly in Africa, with a case fatality rate of up to 90%. The Ebola virus is transmitted to humans from wild animals, and human-to-human transmission occurs through direct contact with bodily fluids or contaminated surfaces. Clinical diagnosis of Marburg virus disease (MVD) can be difficult. Many of the signs and symptoms of MVD are similar to other infectious diseases (such as malaria, typhoid fever, or dengue) or other viral hemorrhagic fevers that may be endemic in the area (such as Lassa fever or EVD). The incubation period of Filoviruses ranges from 2 to 21 days, and symptoms include sudden onset fever, fatigue, muscle pain, headache, and sore throat, followed by vomiting, diarrhea, rash, impaired kidney and liver function, and sometimes internal and external bleeding (Torreele et al. (2023) The Lancet Infections Diseases Volume 23, Issue 7, E253-E258).

Current challenges for treating hemorrhagic fever infections include limited therapeutic options, difficulty in early diagnosis, rapid disease progression, high mortality rates, and the need for strict isolation and infection control measures during treatment and outbreaks. Existing treatments for hemorrhagic fever infections primarily focus on supportive care to manage symptoms or palliative care. There is a need for new therapeutic compounds that can effectively treat hemorrhagic fever infections, reduce the severity of symptoms, and decrease mortality rates.

### SUMMARY

The present disclosure relates in several embodiments to peptoid compounds, compositions and method of use thereof for treating or preventing replication of a virus of the Filoviridae family, and for treating or preventing an infection of a virus of the Filoviridae family in a subject.

According to a first aspect, the present disclosure relates in several embodiments to a method of treating or preventing an infection of a virus of the Filoviridae family in a subject. The method comprises administering to the subject an effective amount of one or more peptoid compounds selected from: a peptoid compound H-Ndec-NLys-Nspe-Nspe(p-Br)-NLys-Npse-Nspe(p-Br)-NH₂, a peptoid compound H-Ndec-(NLys-Nspe-Nspe(p-Br))₂-NLys-NH₂, and a combination thereof, thereby treating or preventing the infection.

The method of treating or preventing an infection of a virus of the Filoviridae family in a subject may include the following details, which can be combined with one another in any combinations unless clearly mutually exclusive:
(i) The virus may be Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.
(ii) The administering may be intranasal, oral, transdermal, transmucosal, intraperitoneal, subcutaneous, intramuscular, or intravenous, or any combinations thereof.
(iii) The effective amount may be from 1 mg/day to 1000 mg/day, or from 25 mg/day to 750 mg/day, or from 50 mg/day to 500 mg/day, or from 100 mg/day to 400 mg/day.
(iv) The administering may be once per day, twice per day, three times per day, four times per day, once per week, twice per week, once per two weeks, or once per month.

According to a second aspect, the present disclosure relates in several embodiments to a method of inhibiting or preventing replication of a virus of the Filoviridae family. The method comprises: contacting the virus with a peptoid compound H-Ndec-NLys-Nspe-Nspe(p-Br)-NLys-Npse-Nspe(p-Br)-NH₂, a peptoid compound H-Ndec-(NLys-Nspe-Nspe(p-Br))₂-NLys-NH₂, a peptoid compound H-NLys-NSpe(p-Br)-NSpe(p-Br)-NLys-NSpe(p-Br)₅-NSpe(p-Br)-NH₂, a peptoid compound H-Ntetradec-NLys-Nspe-Nspe-NLys-NH₂, or any combination thereof, in an amount and for a time sufficient to inhibit or prevent replication of the virus.

The method of inhibiting or preventing replication of a virus of the Filoviridae family may include the following details, which can be combined with one another in any combinations unless clearly mutually exclusive:
(i) The virus may be Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.
(ii) The method may include binding the one or more peptoid compounds to a surface of an object, thereby conferring antiviral activity to the object.
(iii) The method may include applying a composition comprising the peptoid to a surface of an object.
(iv) The method may include applying a composition comprising the peptoid as a cleaning solution to a subject's skin.

### BRIEF DESCRIPTION OF THE DRAWINGS

The present disclosure may be further understood through reference to the attached figures in combination with the detailed description that follows.

FIG. 1 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid MXB-24,656, following 2 hour incubation of the EBOV-GFP with the peptoid MXB-24,656 at 20 μg/mL or 100 μg/mL. Micrograph images labeled E, F, G, and H respectively show results of GFP fluorescence imaging at 24 hours, 48 hours, 72 hours, and 96 hours after the indicated treatment of 20 μg/mL. Similarly, micrograph images labeled M, N, O, and P respectively show results of GFP fluorescence imaging at 24 hours, 48 hours, 72 hours, and 96 hours after the indicated treatment 100 μg/mL. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 2 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-22,510, following 1 hour incubation of the EBOV-GFP with the peptoid compound MXB-22,510 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 3 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-27,369, following 1 hour incubation of the EBOV-GFP with the peptoid compound MXB-27,369 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 4 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-24,605, following 1 hour incubation of the EBOV-GFP with the peptoid compound MXB-24,605 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 5 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-24,816, following 1 hour incubation of the EBOV-GFP with the peptoid compound MXB-24,816 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 6 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-25,739, following 1 hour incubation of the EBOV-GFP with the peptoid compound MXB-25,739 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 7 is a set of example micrographs of cultured VeroE6 cells in 24-well plate wells at 24 hours, 48 hours, 72 hours, and 96 hours after treatment with a virus-peptoid mixture comprising a suspension of transgenic GFP-expressing Ebola virus (EBOV-GFP) mixed with peptoid compound MXB-21,008, following 1 hour incubation of the EBOV-GFP with the peptoid compound MXB-21,008 at 20 μg/mL or 100 μg/mL. Micrograph images labeled E, F, G, and H respectively show results of GFP fluorescence imaging at 24 hours, 48 hours, 72 hours, and 96 hours after the indicated treatment. Similarly, micrograph images labeled M, N, O, and P respectively show results of GFP fluorescence imaging at 24 hours, 48 hours, 72 hours, and 96 hours after the indicated treatment. Micrograph images labeled A, B, C, and D respectively show example brightfield images of the cells at 24 hours, 48 hours, 72 hours, and 96 hours after the indicated treatment. Similarly, micrograph images labeled I, J, K, and L respectively show example brightfield images of the cells at 24 hours, 48 hours, 72 hours, and 96 hours after the indicated treatment.

FIG. 8 shows in micrograph images labeled A-H a set of example micrographs of cultured VeroE6 cells in 24-well plate wells at 24 hours, 48 hours, 72 hours, and 96 hours after treatment with a suspension of transgenic GFP-expressing Ebola virus (EBOV-GFP) that had not been incubated with a peptoid compound (“No inhibitor”). Micrograph images labeled A, B, C, and D respectively show example brightfield images of the cells at 24 hours, 48 hours, 72 hours, and 96 hours after the indicated treatment. Micrograph images labeled E, F, G, and H respectively show results of GFP fluorescence imaging at 24 hours, 48 hours, 72 hours, and 96 hours after the indicated treatment.

FIG. 8 also shows in micrograph images labeled I-P a set of example micrographs of cultured VeroE6 cells in 24-well plate wells at 24 hours, 48 hours, 72 hours, and 96 hours timepoints. The cultured VeroE6 cells in I-P (“uninfected”) were not treated with any peptoid compound and were not infected with transgenic GFP-expressing Ebola virus (EBOV-GFP). Micrograph images labeled I, J, K, and L respectively show example brightfield images of the cells at 24 hours, 48 hours, 72 hours, and 96 hours. Micrograph images labeled M, N, O, and P respectively show results of GFP fluorescence imaging at 24 hours, 48 hours, 72 hours, and 96 hours after the indicated treatment.

FIG. 9 is a graph reporting example quantification of titers of transgenic GFP-expressing Ebola virus (EBOV-GFP) in cell culture supernatants removed at time points 0, 24, 48, 72, and 96 hours post-infection of VeroE6 cells treated with a virus-peptoid mixture comprising a suspension of EBOV-GFP mixed with peptoid compounds A, B, C, D, E, F, or G, following 2 hours incubation of the EBOV-GFP with the peptoid compound at 20 μg/mL. In addition, one group of cultured VeroE6 cells was treated with EBOV-GFP that had not been incubated with a peptoid compound (“no inhibitor”), and one group of cultured VeroE6 cells was not treated with any peptoid compound and was not infected with transgenic EBOV-GFP (“uninfected”).

FIG. 10 is a graph reporting example quantification of titers of transgenic GFP-expressing Ebola virus (EBOV-GFP) in cell culture supernatants removed at time points 0, 24, 48, 72, and 96 hours post-infection of VeroE6 cells treated with a virus-peptoid mixture comprising a suspension of EBOV-GFP mixed with peptoid compounds MXB-24,656; MXB-22,510; MXB-27,369; MXB-24,605; MXB-24,816; MXB-25,739 or MXB-21,008 following 2 hours incubation of the EBOV-GFP with the peptoid compound at 100 μg/mL. In addition, one group of cultured VeroE6 cells was treated with EBOV-GFP that had not been incubated with a peptoid compound (“no inhibitor”).

FIG. 11 is the molecular structure of peptoid compound MXB-24,656.

FIG. 12 is the molecular structure of peptoid compound MXB-22,510.

FIG. 13 is the molecular structure of peptoid compound MXB-27,369.

FIG. 14 is the molecular structure of peptoid compound MXB-25,605.

FIG. 15 is the molecular structure of peptoid compound MXB-24,816.

FIG. 16 is the molecular structure of peptoid compound MXB-25,739.

FIG. 17 is the molecular structure of peptoid compound MXB-21,008.

FIG. 18 is a Table reporting the example data that are presented in the graph in FIG. 9.

FIG. 19 is a Table reporting the example data that are presented in the graph in FIG. 10.

### DETAILED DESCRIPTION

In some embodiments, the present disclosure relates to a method of treating a subject for an infection of a virus of the Filoviridae family. The method comprises administering to the subject an effective amount of a peptoid compound described herein, thereby treating the subject for the infection. In some embodiments, the present disclosure relates to peptoid compounds for treating hemorrhagic fever diseases caused by Filoviridae infection.

In some embodiments, the present disclosure relates to methods of inhibiting or preventing replication of a virus of the Filoviridae family.

Filoviridae is a family of viruses that includes several dangerous pathogens, most notably Ebola virus and Marburg virus. These viruses are responsible for severe and often fatal hemorrhagic fever in humans and other mammals, including but not limited to non-human primates. The family Filoviridae is part of the order Mononegavirales. Filoviruses are filamentous, enveloped viruses with a non-segmented, single-stranded RNA genome. They are characterized by their distinctive thread-like or filamentous shape under electron microscopy. The viruses in this family are known for causing severe and often fatal diseases with high mortality rates, particularly in sub-Saharan Africa. Ebola virus gained international attention due to several outbreaks in Africa, with the most notable ones occurring in the late 20^{th} and early 21^{st} centuries. Marburg virus is another member of the Filoviridae family and is associated with outbreaks, although it is less common than Ebola virus. These viruses are transmitted to humans from wild animals and can then spread through human-to-human transmission. The natural reservoirs of filoviruses are believed to be certain species of bats, and the viruses can be transmitted to humans through the handling or consumption of infected animals.

The Filoviridae family includes, without limitation, the following viruses: Lloviu virus (also known by the abbreviation LLOV, or the species name Lloviu cuevavirus) in the Cuevavirus genus; Měnglà virus (also known by the abbreviation MLAV, or the species name Mengla dianlovirus) in the Dianlovirus genus; members of the Ebolavirus genus including without limitation Bombali virus (also known by the abbreviation BOMV, or the species name Bombali ebolavirus), Bundibugyo virus (also known by the abbreviation BDBV, previously known as BEBOV, or the species name Bundibugyo ebolavirus), Reston virus (also known by the abbreviation RESTV, previously REBOV, or the species name Reston ebolavirus), Sudan virus (also known by the abbreviation SUDV, previously SEBOV, or the species name Sudan ebolavirus), Taï Forest virus (also known by the abbreviation TAFV, previously CIEBOV, or the species name Taï Forest ebolavirus), or Ebola virus (also known by the abbreviation EBOV, previously ZEBOV, or the species name Zaire ebolavirus); members of the Marburgvirus genus including without limitation Marburg virus (also known by the abbreviation MARV, or the species name Marburg marburgvirus), or Ravn virus (also known by the abbreviation RAVV); members of the Striavirus genus including without limitation Xīlǎng virus (also known by the abbreviation XILV, or the species name Xilang striavirus); and members of the Thamnovirus genus including without limitation Huángjiāo virus (also known by the abbreviation HUJV or the species name Huangjiao thamnovirus).

In some embodiments, the virus may be Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.

In some embodiments, the infection may be undiagnosed, diagnosed, suspected, or confirmed. In some embodiments, the administering is to prevent an infection.

Diagnosing a Filoviridae family virus infection in a subject typically involves a combination of clinical, epidemiological, and laboratory criteria. For example, diagnosing Ebola virus disease may involve the following methods: 1. Clinical Presentation: Symptoms and Signs: Clinicians consider the subject's symptoms, which often include fever, severe headache, muscle pain, fatigue, diarrhea, vomiting, abdominal pain, and unexplained bleeding or bruising. Epidemiological History: In regions where Ebola is endemic or during outbreaks, a subject's travel history and potential exposure to the virus (contact with infected individuals or animals) may be considered for diagnosis. 2. Laboratory Tests: Real-Time Reverse Transcription Polymerase Chain Reaction (RT-PCR) may be used for diagnosing Ebola virus, by detecting the virus's genetic material in blood, serum, or other body fluids. Samples are typically collected from subjects with symptoms consistent with Ebola virus disease. Antigen Detection Tests: Rapid diagnostic tests that detect Ebola virus antigens in blood samples can provide quicker results than RT-PCR. However, they may be less sensitive and specific compared to PCR. Virus Isolation: Culturing the virus from subject samples is a more complex and time-consuming method. 3. Serological Tests: IgM/IgG Antibody Detection: Serological tests can detect the presence of Ebola virus-specific antibodies (e.g., IgM and IgG) in the subject's blood. However, these tests may take time to become positive after the onset of symptoms. 4. Point-of-Care Tests: RDTs (Rapid Diagnostic Tests): Some rapid diagnostic tests have been developed for use in the field, providing quicker results at the point of care. These tests can detect the virus or antibodies in blood or serum. 5. Differential Diagnosis: Exclusion of Other Diseases: Since the symptoms of Ebola virus disease overlap with those of other infectious diseases (such as malaria, typhoid fever, and other viral hemorrhagic fevers), differential diagnosis may be performed.

The terms “peptoid” or “peptoid compound” as used herein refers to a type of biomimetic molecule that is similar to peptides but differs in its structure. Peptoids are synthetic oligomers composed of N-substituted glycine units. Accordingly, peptoids are also known as poly-N-substituted glycine compounds. In contrast to peptides, which have a peptide bond between amino acids, peptoids have an N-substituted (or N-alkylated) amide bond. This structural difference gives peptoids unique properties compared to peptides. Peptoids can be designed and synthesized to mimic the functions of natural peptides but with enhanced stability and different chemical properties. Peptoid compounds may be cyclic or linear. Peptoids have been described, for example, in U.S. Pat. Nos. 8,445,632, 8,828,413, 9,315,548, 9,872,495, 9,938,321, and International Patent Application Publication No.'s WO2021046562, WO2020223581, WO2021127294, WO2023287570, WO2022120393, and WO2021231343, the disclosures of which are incorporated herein in their entireties. For example, without limitation, in some embodiments a peptoid compound may have a formula:

A-(X—Y—Z)ₙ-B.

In such a compound, A can be selected from H and a terminal N-alkyl substituted glycine residue, where such an alkyl substituent can be selected from about C4 to about C20 linear, branched and cyclic alkyl moieties; n can be an integer selected from 1-3; B can be selected from NH₂, and one and two N-substituted glycine residues, such N-substituents as can be independently selected from a-amino acid sidechain moieties and structural/functional analogs thereof; and X, Y and Z can also be independently selected from N-substituted glycine residues, such N-substituents as can be independently selected from α-amino acid sidechain moieties and structural/functional analogs thereof and proline residues. Such X—Y—Z periodicity can provide such a compound a certain amphipathicity. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain—such homologs as would be understood in the art, including but not limited to plus or minus 1 or 2 or more methylene and/or methyl groups.

A can be H, and B can be selected from one or two N-substituted glycine residues, such a selection can reduce the hydrophobicity of such a compound, as compared to compounds of 3-fold periodicity. In certain such embodiments, X can be an Nlys residue; n can be 2-3; and B can be two N-substituted glycine residues. Without limitation, such a compound can be of a formula:

H-(N_{Lys}-Nₛₚₑ-Nₛₚₑ)₃-N_{Lys}-Nₛₚₑ-NH₂.

Regardless of the identity of A, X and B, at least one of Y and Z can be a proline residue. X, Y and Z can be proline residues.

In certain other embodiments, A can be a terminal N-alkyl substituted glycine residue, with such an alkyl substituent as can be selected from about C6 to about C18 linear alkyl moieties. Regardless, B can be NH2, and n can be selected from 1 and 2. In certain such embodiments, A can be a terminal N-alkyl substituted glycine residue, with an alkyl substituent selected from about C6 to about C18 linear alkyl moieties. Regardless, B can be an Nlys residue, and n can be 1.

In some embodiments, a peptoid compound may have a formula:

H-(N_{Lys}-Y—Z)ₙ-Y′—Z′—NH₂

wherein n can be selected from 2 and 3; and Y, Z, Y′ and Z′ can be independently selected from N-substituted glycine residues, where such substituents can be independently selected from α-amino acid sidechain moieties and carbon homologs thereof. Such Y′ and Z′ residues can be selected to provide such compound reduced hydrophobicity as compared to a compound of 3-fold periodicity. In certain such embodiments, at least one of X and Y can be a proline residue. Regardless, n can be selected from 2 and 3, and Y′ can be an N_{Lys} residue. In certain such embodiments, one or both X and Y can be proline residues. Without limitation, such a compound with reduced hydrophobicity can be of a formula:

H-(N_{Lys}-Nₛₚₑ-Nₛₚₑ)₃-N_{Lys}-Nₛₚₑ-NH₂

In some embodiments, a peptoid compound may have a formula:

H-N_{R}-(X—Y—Z)ₙ-B

wherein B can be selected from NH₂ and X′; X, Y, Z and X′ can be independently selected from N-substituted glycine residues, where such substituents can be independently selected from α-amino acid sidechain moieties and carbon homologs thereof; n can be an integer selected from 1 and 2; and R can be an N-alkyl substituent of such a glycine residue, as can be selected from about C₄ to about C₂₀ linear, branched and cyclic alkyl moieties. In some embodiments, n can be 2, and B can be NH₂. In some embodiments, n can be 1, and B can be X′. Accordingly, one or both of X and X′ can be N_{Lys} residues. Regardless, an alkyl substituent can be selected from about C₆ to about C₁₈ linear, branched and cyclic alkyl moieties, and X and X′ can be N_{Lys} residues. Without limitation, such a compound can be of a formula:

H-N_{tridec}-N_{Lys}-Nₛₚₑ-Nₛₚₑ-N_{Lys}-NH₂.

A peptoid may be a poly-N-substituted glycine compound comprising an N-terminus selected from H and an N-alkyl substituted glycine residue, where such an alkyl substituent can be selected from about C₄ to about C₂₀ linear, branched and cyclic alkyl moieties; a C-terminus selected from NH₂, one and two N-substituted glycine residues, such N-substituents as can be independently selected from a-amino acid sidechain moieties and structural/functional analogs thereof; and 2 to about 15 monomeric residues between the N-and C-termini, each such residue as can be independently selected from proline residues and N-substituted glycine residues, said N-substituents independently selected from α-amino acid sidechain moieties and structural/functional analogs thereof. Such monomers can be selected to provide such a compound a non-periodic sequence of monomers. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such sidechain—such homologs as would be understood by those skilled in the art, including but not limited to plus or minus 1 or 2 or more methylene and/or methyl groups.

The N-terminus of such a compound can be H; and the C-terminus can be selected from said one and two N-substituted glycine residues. A peptoid compound can comprise 2 to about 5 (X—Y—Z) non-periodic trimers. At least one of X, Y and Z in each of the trimers can be selected to interrupt 3-fold periodicity. Without limitation, at least one X in at least one said trimer can be an Nlys residue. At least one of Y and Z in at least one such trimer can be a proline residue. The monomeric residues can comprise at least two non-consecutive of the same or repeat trimers, with at least one such residue therebetween to interrupt periodicity. At least one X in at least one such trimer can be an Nlys residue, and at least one of Y and Z in at least one said trimer can be a proline residue.

The N-terminus of such a compound can be an N-alkyl substituted glycine residue, with an alkyl substituent selected from about C6 to about C18 linear alkyl moieties. A peptoid compound can comprise 2 to about 5 (X-Y-Z) non-periodic trimers. At least one of X, Y and Z in each of the trimers can be selected to interrupt 3-fold periodicity. The monomeric residues can comprise at least two non-consecutive of the same or repeat trimers, with at least one residue therebetween to interrupt periodicity. At least one X in at least one said trimer can be an Nlys residue, and at least one of Y and Z in at least one said trimer can be a proline residue.

Various halogenated peptoids may be utilized in accordance with the teachings herein to make antiviral pharmaceutical compositions and treatments. These include, without limitation, various halogenated analogs of the foregoing peptoid compounds. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. In some embodiments, the use of one or two bromo-or chloro-substitutions may be used. The peptoids described herein may be halogenated at various locations, for example and without limitation para halogenation on the peptoids containing aryl rings, ortho- and meta-substitution, or perhalogentation.

The peptoids described herein may be alkylated, for example and without limitation terminal alkylation. For example and without limitation, the alkyl substituent may be selected from about C₆ to about C₁₈ linear alkyl moieties.

In some embodiments, a peptoid may have antiviral activity. Without wishing to be bound by theory, antiviral activity of a peptoid may be associated with its ability to bind to or pass through a viral membrane and to bind to or destroy viral DNA and/or RNA.

In some embodiments, a peptoid may have antiviral activity against a virus of the Filoviridae family. The antiviral activity of a particular peptoid against a virus of the Filoviridae family may be unpredictable. A particular peptoid's antiviral activity against a virus of the Filoviridae family may be determined empirically, for example by testing a particular peptoid for antiviral activity in an in vitro assay or an in vivo assay. Without being bound by a particular theory, if a peptoid is shown to have antiviral activity against an example virus of the Filoviridae family, it is contemplated that the peptoid will have similar antiviral activity against other viruses of the Filoviridae family. For example, without intending to be limiting, if a peptoid shows antiviral activity against Ebola virus, it is contemplated that the peptoid will have antiviral activity against other viruses of the Ebolavirus genus. As a further example, without intending to be limiting, if a peptoid shows antiviral activity against Ebola virus, it is contemplated that the peptoid will have antiviral activity against other viruses of the Filoviridae family.

Various peptoid compounds may be utilized in accordance with the teachings herein to make antiviral pharmaceutical compositions and treatments. In addition to the peptoids set forth in Table 1, these include the peptoids described in the various patents and patent application publications described herein, which are incorporated herein in their entirety.

The peptoids described herein may be synthesized and provided by any suitable method known in the art, such as, for example and not by way of limitation, the method described in Example 1 of the present disclosure, or by methods described in the patents and patent application publications disclosed herein.

Various counterions may be utilized in forming pharmaceutically acceptable salts of the peptoids disclosed herein. In some embodiments, pharmaceutically acceptable salts of the peptoids disclosed herein may include Acetate, Aspartate, Benzenesulfonate, Benzoate, Besylate, Bicarbonate, Bitartrate, Bromide, Camsylate, Carbonate, Chloride, Citrate, Decanoate, Edetate, Esylate, Fumarate, Gluceptate, Gluconate, Glutamate, Glycolate, Hexanoate, Hydroxynaphthoate, Iodide, Isethionate, Lactate, Lactobionate, Malate, Maleate, Mandelate, Mesylate, Methylsulfate, Mucate, Napsylate, Nitrate, Octanoate, Oleate, Pamoate, Pantothenate, Phosphate, Polygalacturonate, Propionate, Salicylate, Stearate, Acetate, Succinate, Sulfate, Tartrate, Teoclate, Tosylate salts.

In some embodiments, the present disclosure extends to the preparation of prodrugs and derivatives of the peptoids of the invention. Prodrugs are derivatives which have cleavable groups and become by solvolysis or under physiological conditions the peptoid of the invention, which are pharmaceutically active. Such examples include, but are not limited to, choline ester derivatives and the like, N-alkyl morpholine esters and the like. In some embodiments, the peptoid compounds provided herein may be prepared e.g., in crystalline form and may be solvated or hydrated. Suitable solvates include pharmaceutically acceptable solvates, such as hydrates, and further include both stoichiometric solvates and non-stoichiometric solvates.

In some embodiments, the peptoid compound is referred to herein as “MXB-27,369” having a formula H-Ndec-Nlys-Nspe-Nspe(p-Br)-Nlys-Npse-Nspe(p-Br)-NH₂, and having a molecular structure:

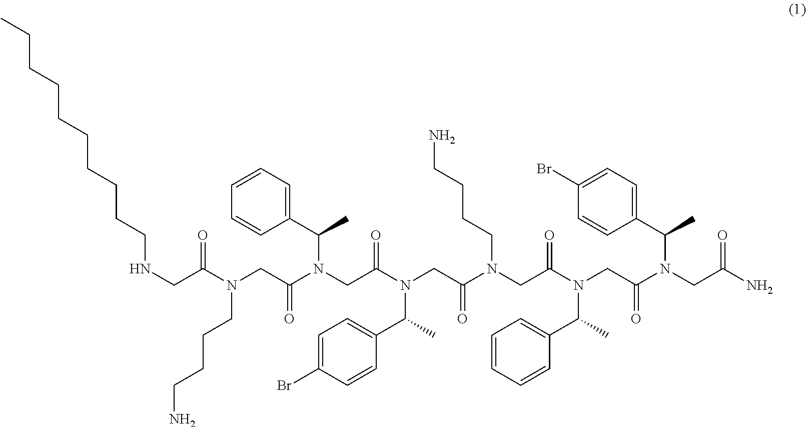 [SMILES: CCCCCCCCCCNCC(=O)N(CCCCN)CC(=O)N(CC(=O)N(CC(=O)N(CCCCN)CC(=O)N(CC(=O)N(CC(N)=O)[C@H](C)c1ccc(Br)cc1)[C@H](C)c1ccccc1)[C@H](C)c1ccc(Br)cc1)[C@H](C)c1ccccc1]

In some embodiments, the peptoid compound is referred to herein as “MXB-25,739” having a formula H-Ndec-(Nlys-Nspe-Nspe(p-Br))₂-Nlys-NH₂ and having a molecular structure:

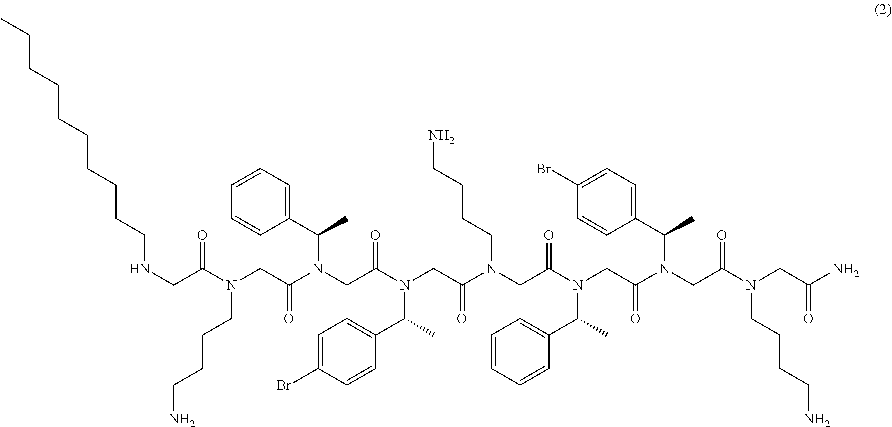 [SMILES: CCCCCCCCCCNCC(=O)N(CCCCN)CC(=O)N(CC(=O)N(CC(=O)N(CCCCN)CC(=O)N(CC(=O)N(CC(=O)N(CCCCN)CC(N)=O)[C@H](C)c1ccc(Br)cc1)[C@H](C)c1ccccc1)[C@H](C)c1ccc(Br)cc1)[C@H](C)c1ccccc1]

In some embodiments, the peptoid compound is referred to herein as “MXB-24,656” having a formula H-Nlys-Nspe(p-Br)-Nspe(p-Br)-Nlys-Nspe(p-Br)₅-Nspe(p-Br)-NH₂ and having a molecular structure:

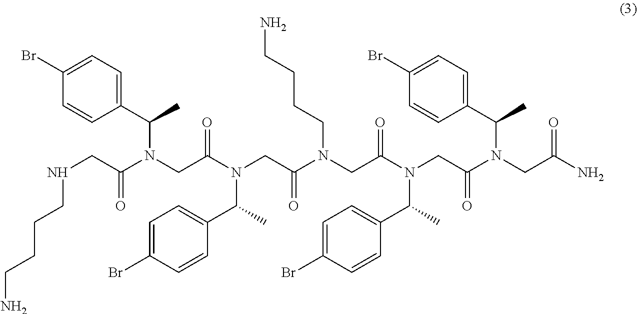 [SMILES: C[C@H](c1ccc(Br)cc1)N(CC(N)=O)C(=O)CN(C(=O)CN(CCCCN)C(=O)CN(C(=O)CN(C(=O)CNCCCCN)[C@H](C)c1ccc(Br)cc1)[C@H](C)c1ccc(Br)cc1)[C@H](C)c1ccc(Br)cc1]

In some embodiments, the peptoid compound is referred to herein as “MXB-24,816” having a formula H-Ntetradec-Nlys-Nspe-Nspe-Nlys-NH₂ and having a molecular structure:

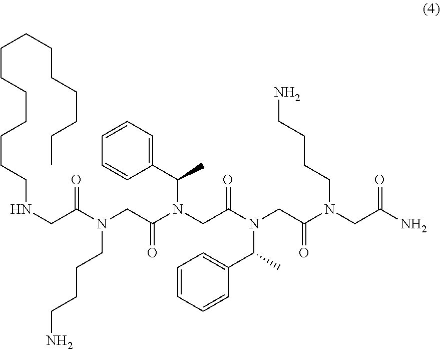 [SMILES: CCCCCCCCCCCCCCNCC(=O)N(CCCCN)CC(=O)N(CC(=O)N(CC(=O)N(CCCCN)CC(N)=O)[C@H](C)c1ccccc1)[C@H](C)c1ccccc1]

As described in the Example 2 of the present disclosure, it has been surprisingly found that four of the 7 peptoid compounds tested selectively showed antiviral activity against Ebola virus in infected cells in vitro.

In particular, two peptoid compounds, MXB-27,369 and MXB-25,739 showed antiviral activity against Ebola virus following incubation at a low concentration (20 μg/mL) with Ebola virus. In addition, four peptoid compounds, MXB-27,369, MXB-25,739, MXB-24,656, and MXB-24,816 showed antiviral activity against Ebola virus following incubation at a higher concentration (100 μg/mL) with Ebola virus.

Also as described in Example 2 of the present disclosure, none of the other peptoid compounds tested showed antiviral activity against Ebola virus. The observed selective antiviral activity of peptoid compounds MXB-27,369, MXB-25,739, MXB-24,656, and MXB-24,816 (and not the other peptoid compounds tested) against Ebola virus was not predictable in view of the knowledge in the art.

In some embodiments, antiviral activity may refer to preventing, inhibiting or decreasing replication of a virus.

In some embodiments, the peptoids disclosed herein may be effective to treat a subject for an infection involving at least one virus of the Filoviridae family.

In some embodiments, one or more peptoid compounds described herein can be used in pharmaceutical compositions for administration to a subject, to prevent or treat an infection of a virus of the Filoviridae family.

In some embodiments, one or more peptoids compounds suitable for use in pharmaceutical compositions for administration to a subject may include peptoid compound MXB-27,369, peptoid compound MXB-25,739, or a combination thereof.

In some embodiments, the present disclosure relates to a method of treating a subject for an infection of a virus of the Filoviridae family. The method comprises administering to the subject an effective amount of one or more peptoid compounds selected from: a peptoid compound MXB-27,369 having a formula H-Ndec-Nlys-Nspe-Nspe(p-Br)-Nlys-Npse-Nspe(p-Br)-NH₂, a peptoid compound MXB-25,739 having a formula H-Ndec-(Nlys-Nspe-Nspe(p-Br))₂-Nlys-NH₂, or a combination thereof, thereby treating the subject for the infection.

The one or more peptoids may be effective in simultaneously treating infections involving the one or more viruses.

In some embodiments, the virus may be Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.

A wide variety of tissues infected with the one or more viruses may be treated with the compositions and methods disclosed herein. These include, without limitation, the tissues of the entire body, internal organs, skin, ear, nose, sinus, throat, mouth, lungs, gastrointestinal tract, muscle, brain and nervous system.

In some embodiments, the administering may be via intranasal, oral, transdermal, transmucosal, intraperitoneal, subcutaneous, intramuscular, or intravenous routes, or any combinations thereof.

In some embodiments, the peptoids and compositions described herein are effective to treat one or more symptoms, such as fever, headache, muscle pain, fatigue, diarrhea, vomiting, abdominal pain, and bleeding or bruising. Treatment includes preventing and/or decreasing symptoms associated with an infection. In some embodiments, the method may include treating a subject susceptible to or afflicted with a condition attributable to or resulting from an infection of a virus of the Filoviridae family, the method comprising administering an effective amount of a pharmaceutical composition comprising a peptoid compound described herein.

“Treating” or “treatment” of a disease refers, in some embodiments, to ameliorating the disease or disorder (e.g., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both.

“Preventing” or “prevention” refers to a reduction in risk of acquiring a disease (e.g., causing at least one of the clinical symptoms of the disease not to develop in a subject not yet exposed to or predisposed or susceptible to the disease, and not yet experiencing or displaying symptoms of the disease).

In some embodiments, the subject may be a vertebrate animal. In some embodiments, the subject may be a mammal. In some embodiments, the subject may be a primate. In some embodiments, the subject may be a human. In some embodiments, the methods disclosed herein have veterinary applications and can be used to treat non-human animals, such as wild, domestic, or farm animals, including, but not limited to, cattle, sheep, goats, pigs, dogs, cats, and poultry.

The peptoid compounds of the present disclosure may be formulated in a composition. Such compositions can be prepared in a manner known in the pharmaceutical art. The peptoid compounds described herein can be formulated into pharmaceutically acceptable compositions and dosage forms for administration to a subject. In some embodiments, the present disclosure relates to a composition comprising an effective amount of a peptoid compound described herein for use in a method of treating a subject for an infection of a pathogen member of the Filoviridae family. In some embodiments, the present disclosure relates to the use of the peptoids described herein for the preparation of medicaments or as medicaments, that may be used for treating an infection of a virus of the Filoviridae family.

The present disclosure provides pharmaceutical compositions comprising one or more peptoids and a pharmaceutically acceptable medium, such as an excipient, carrier, or the like. The peptoids described herein may be dissolved, suspended or dispersed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

Generally, the peptoid compounds described herein are administered in a therapeutically effective amount. “Therapeutically effective amount” means the amount of a compound that, when administered to a subject for treating a disease, is sufficient to effect such treatment for the disease. The therapeutically effective amount of the peptoid compound may be determined by a physician, in the light of the relevant circumstances, including the condition to be treated, the chosen route of administration, the peptoid compound administered, the age, weight, and response of the individual subject, the severity of the subject's symptoms, and the like.

In some embodiments, the effective amount may be from 1-1000 mg/day, with a preferred embodiment of 25-750 mg/day, or a more preferred embodiment of 50-500 mg/day, or an even more preferred embodiment of 100-400 mg/day.

The pharmaceutical compositions disclosed herein may be administered to a subject or applied in various manners. Thus, for example, these compositions may be administered via intranasal, oral, transdermal, transmucosal, intravenous, intraperitoneal, intramuscular, pulmonary, or subcutaneous routes. Moreover, these compositions may be applied in a single dose, multi-dose or controlled release fashion.

In some embodiments, the administration may be one, two, three, or four times per day. Additional embodiments include administration, once every 72 hours, once per week, once per 2 weeks, or once per month.

The pharmaceutical compositions disclosed herein may be manufactured as tablets, liquids, gels, foams, ointments, emulsions, or powders. In some embodiments, these compositions may be applied as microparticles or nanoparticles.

In some embodiments, intranasal compositions may comprise any pharmaceutically acceptable excipient, such as those approved in nasal spray formulations and listed in the Food and Drug Administration's Inactive Ingredient Database, or justifiable based on the Food and Drug Administration's Guidance for Industry: Nasal Spray and inhalation Solution, Suspension, and Spray Drug Products—Chemistry, manufacturing, and Controls Documentation. As would be understood by skilled persons, typically, the excipients used in intranasal formulations should be safe and compatible with nasal mucosa. Some common excipients used in intranasal products include buffers to maintain the pH of the formulation within an acceptable range, preservatives to prevent microbial contamination, surfactants to enhance drug absorption and distribution, stabilizers to maintain the stability of the formulation over time, solubilizers to improve the solubility of poorly soluble drugs, viscosity modifiers to control the viscosity of the formulation for better administration, and tonicity agents to adjust the osmolarity of the formulation to be close to that of nasal mucosa.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for subjects, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the peptoid compound is usually a minor component (e.g., from about 0.01% to about 50% by weight) with the remainder being various vehicles or carriers and processing aids helpful for forming the desired dosing form.

Liquid forms suitable for oral administration may include, without limitation, a suitable aqueous or nonaqueous vehicle with buffers, suspending and dispensing agents, colorants, flavors and the like.

Solid forms may include, without limitation any of the following ingredients, or compounds of a similar nature: a binder such as microcrystalline cellulose, gum tragacanth or gelatin; an excipient such as starch or lactose, a disintegrating agent such as alginic acid, Primogel, or cornstarch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. The peptoid compound in such compositions is typically a minor component, often being from about 0.05% to 10% by weight with the remainder being the injectable carrier and the like.

Transdermal compositions are typically formulated as a topical ointment or cream containing the peptoid compound, generally in an amount ranging from about 0.01 to about 20% by weight. When formulated as an ointment, the peptoid compound may be combined with either a paraffinic or a water-miscible ointment base. The peptoid compound may be formulated in a cream with, for example an oil-in-water cream base. Such transdermal formulations may include additional ingredients to enhance the dermal penetration of stability of the peptoid compounds or the formulation.

The peptoid compounds of the present disclosure can be administered by a transdermal device. Accordingly, transdermal administration can be accomplished using a patch either of the reservoir or porous membrane type, or of a solid matrix variety.

The peptoid compounds of the present disclosure can be administered subcutaneously, including, without limitation, the use of syringe and needle injection, autoinjectors, pen injectors, needle-free injectors, subcutaneous infusion, jet injectors, patch pumps, pump infusion sets, implantable devices, subcutaneous depots, subcutaneous sustained release formulations, or any combinations thereof. The most traditional and widely used method of subcutaneous administration involves using a syringe and needle. Autoinjectors are pre-filled devices that automatically inject a set dose of a pharmaceutical composition when pressed against the skin. Examples include, without limitation, EpiPen for epinephrine and various biologic medications. Similar to autoinjectors, pen injectors are pre-filled devices that allow patients to self-administer a specific dose of a pharmaceutical composition. They are user-friendly and may have features like dose adjustment. Needle-free injectors use high pressure to administer a pharmaceutical composition through the skin without using a needle. Subcutaneous infusion may involve using an infusion pump to deliver a continuous or intermittent flow of a pharmaceutical composition into the subcutaneous tissue. Jet injectors use a high-pressure stream of liquid to penetrate the skin and deliver a pharmaceutical composition into the subcutaneous tissue. Patch pumps may adhere to the skin and contain a reservoir of a pharmaceutical composition, and it is absorbed through the skin over a period of time. Pump infusion sets may include a cannula or needle that is placed under the skin for continuous pharmaceutical composition delivery. Implantable devices may be used to provide sustained release of a pharmaceutical composition subcutaneously. Implantable devices may be surgically implanted and can deliver a controlled dose of a pharmaceutical composition over an extended period.

Subcutaneous depot release refers to the administration of pharmaceutical compositions in a way that allows for sustained and controlled release of a pharmaceutical composition from a depot or reservoir located in the subcutaneous tissue. This method may be used to provide a prolonged therapeutic effect, reducing the frequency of dosing and improving patient compliance. In some embodiments, a subcutaneous depot release formulation may include, without limitation, a solution, a suspension, or biodegradable matrix, that is introduced (e.g.25ypromted) into the subcutaneous tissue. The formulation then forms a depot, a localized reservoir of a pharmaceutical composition, beneath the skin. The subcutaneous depot formulation may release an active substance, e.g. a peptoid, gradually over an extended period.

A subcutaneous depot formulation may include, without limitation, biodegradable matrices, liposomal formulations, polymeric microspheres or nanoparticles, hydrogels, PLGA (poly (lactic-co-glycolic acid)) microparticles, implantable devices, or any combinations thereof. In biodegradable polymers or matrices, over time, the matrix breaks down, releasing a pharmaceutical composition in a controlled manner. Liposomes, which are lipid vesicles, can encapsulate a pharmaceutical composition and provide controlled release. Such liposomal formulations may be injected subcutaneously to create a depot of a pharmaceutical composition. Microspheres or nanoparticles made of biocompatible polymers can encapsulate a pharmaceutical composition and release it slowly over time. These particles can be suspended in a liquid formulation and injected into the subcutaneous tissue. Hydrogels are water-containing gels that can hold and release a pharmaceutical composition. Injectable hydrogels can form depots in the subcutaneous tissue. PLGA microparticles comprise PLGA, a biodegradable polymer commonly used to create microparticles for sustained drug release. PLGA microparticles can be injected subcutaneously to form a depot. Some subcutaneous depot release systems involve implantable devices, such as osmotic pumps or reservoirs. These devices are typically placed under the skin during a minor surgical procedure and provide controlled release of a pharmaceutical composition for an extended period.

Example formulations and methods of sustained release subcutaneous administration of the peptoids and pharmaceutical compositions thereof described herein include those described in the following references, the contents of all of which are incorporated herein in their entireties: Judy Senior, Michael L. Radomsky. (2000). Sustained-Release Injectable Products. Boca Raton: CRC Press; Thambi T, Li Y, Lee DS. Injectable hydrogels for sustained release of therapeutic agents. J Control Release. 2017 Dec. 10;267:57-66. Doi: 10.1016/j.jconrel.2017.08.006. Epub 2017 Aug. 4. PMID: 28827094.; Chan YP, Meyrueix R, Kravtzoff R, Nicolas F, Lundstrom K. Review on Medusa: a polymer-based sustained release technology for protein and peptide drugs. Expert Opin Drug Deliv. 2007 July;4(4):441-51. Doi: 10.1517/17425247.4.4.441. PMID: 17683256.; Lou H, Feng M, Hageman MJ. Advanced Formulations/Drug Delivery Systems for Subcutaneous Delivery of Protein-Based Biotherapeutics. J Pharm Sci. 2022 November;111(11):2968-2982. Doi: 10.1016/j.xphs.2022.08.036. Epub 2022 Sep. 2. PMID: 36058255; Sequeira JAD, Santos AC, Serra J, Estevens C, Seiça R, Veiga F, Ribeiro AJ. Subcutaneous delivery of biotherapeutics: challenges at the injection site. Expert Opin Drug Deliv. 2019 February;16(2):143-151. Doi: 10.1080/17425247.2019.1568408. Epub 2019 Jan. 24. PMID: 30632401; Badkar AV, Gandhi RB, Davis SP, LaBarre MJ. Subcutaneous Delivery of High-Dose/Volume Biologics: Current Status and Prospect for Future Advancements. Drug Des Devel Ther. 2021 Jan. 13;15:159-170. Doi: 10.2147/DDDT.S287323. PMID: 33469268; PMCID: PMC7812053; Vaishya R, Khurana V, Patel S, Mitra AK. Long-term delivery of protein therapeutics. Expert Opin Drug Deliv. 2015 March;12(3):415-40. Doi: 10.1517/17425247.2015.961420. Epub 2014 Sep. 24. PMID: 25251334; PMCID: PMC4605535; Remington's Pharmaceutical Sciences, 17^{th} edition, 1985, Mack Publishing Company, Easton, Pa. The above-described components for intranasal, orally administrable, injectable subcutaneous, or topically administrable compositions are merely representative. Other materials as well as processing techniques and the like that are suitable for administering the peptoids and pharmaceutical compositions described herein are identifiable by skilled persons upon reading the present disclosure.

The peptoid compounds described herein can be administered 26ypromened release forms or from sustained release or controlled drug delivery systems, delivered via oral, intramuscular, subcutaneous, or transdermal route. A description of representative sustained release materials and description of delivery systems can be found in Remington's Pharmaceutical Sciences and Modern Pharmaceutics.

In some embodiments, the formulations described herein may include one or more chelation agents. In some embodiments, the chelation agent may be an efficacious anti-calculus agent including, but not limited to, one or more of zinc, hexametaphosphates, and diphosphonates. In some embodiments, the formulations described herein may include one or more chelation agents selected from aminopolycarboxylic acids, citric acid, edetate disodium anhydrous, edetate calcium disodium anhydrous citrate salts, sodium gluconate, transferrins, polymers, and any combinations thereof. In some embodiments, the aminopolycarboxylic acids may be selected from the group consisting of tetraxetan (DOTA), nitrilotriacetic acid (NTA), Ethylenediaminetetraacetic acid (EDTA or EDTA acid), ethylene glycol-bis(β-aminoethyl ether)-N,N,N′,N′-tetraacetic acid (EGTA or egtazic acid), 1,2-bis(o-aminophenoxy)ethane-N,N,N′,N′-tetraacetic acid (BAPTA), pentetic acid, diethylenetriaminepentaacetic acid (DTPA) nicotianamine, ethylenediamine-N,N′-bis(2 hydroxyphenylacetic acid) (EDDHA), Ethylenediamine-N,N′-disuccinic acid (EDDS), and any combinations thereof.

The following non-limiting formulation examples illustrate representative pharmaceutical compositions that may be prepared in accordance with the present disclosure.
Formulation 1—Tablets. A compound of the present disclosure may be admixed as a dry powder with a dry binder in an approximate 1:2 weight ratio. Additional diluent may be added as necessary, and a minor amount of magnesium stearate is added as a lubricant. The mixture is formed into 150-1500 mg tablets (50-500 mg of active compound per tablet) in a tablet press.
Formulation 2—Capsules. A peptoid compound described herein may be admixed as a dry powder with a starch diluent in an approximate 1:1 weight ratio. The mixture is filled into empty capsule shells (50-500 mg of peptoid compound per capsule).
Formulation 3—Liquid. A peptoid compound described herein (50-500 mg) may be admixed with sucrose (1.75 g) and xanthan gum (4 mg) and the resultant mixture may be blended, passed through a No. 10 mesh U.S. sieve, and then mixed in water. Sodium benzoate (10 mg), flavor, and color are diluted with water and added with stirring. Sufficient water may then be added to produce a total volume of 5 mL.
Formulation 4—Tablets. A peptoid compound described herein may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.
Formulation 5—Injection. A peptoid compound described herein may be dissolved or suspended in a buffered sterile saline injectable aqueous medium to a concentration of approximately 0.1-5 mg/mL.
Formulation 6—Topical. Stearyl alcohol (250 g) and a white petrolatum (250 g) may be melted at about 75° C. and then a mixture of a peptoid compound described herein (1-100 g g) methylparaben (0.25 g), propylparaben (0.15 g), sodium lauryl sulfate (10 g), and propylene glycol (120 g) dissolved in water (about 370 g) may be added and the resulting mixture is stirred until it congeals.
Formulation 7—Intranasal. To prepare 1 L of a 25 mM phosphate buffer, dissolve 0.6 g of potassium phosphate dibasic and 2.93 g of potassium phosphate monobasic in 800 mL of deionized (DI) water. Second, slowly add 2 g of both glycerin and 2 g sorbitol while mixing with an overhead mixer. Stir until the solution is clear and free from undissolved particulates. Next, slowly add 50 g of 28ypromellose while stirring with an overhead mixer until dissolved for a 5% solution. Then, add 1 g of EDTA as a preservative for the multidose solution, and finally add 100 mg of the peptoid for a 0.01% (% w/v) solution. The solution is diluted to 1 L with DI water, the pH adjusted with a 1N NaOH or 1N HCl solution to a pH of 5.0-7.0 and stirred until a clear solution is obtained.

The peptoid may be included in the formulation over a range of 0.005%-5%. Alternate buffer agents include histidine buffer for pH control in the physiological range, and may be utilized over a molarity range of 10 mM-100 mM. Alternate viscosity increasing agents include, but are not limited to, carbomers, polyvinylpyrrolidone (PVP), hydroxyethylcellulose (HEC), and poloxamers, and may be present in a range of 2-10%. Osmolality increasing agents can also include, but are not limited to, sorbitol, sodium citrate, or dextrose, and may be included at 1-5%. Taste masking agents can include, but are not limited to, sucrose and/or other sugars and may be present at 1-5%. Preservatives may be included in the range of 0.05%-2%, and can also include, but are not limited to, benzalkonium chloride and sodium benzoate.
Formulation 8—Subcutaneous Injection. To prepare 1 L of a 25 mM phosphate buffer, dissolve 0.6 g of potassium phosphate dibasic and 2.93 g of potassium phosphate monobasic in 800 mL of deionized (DI) water. The solution is diluted to 1 L with DI water, the pH adjusted with a 1N NaOH or 1N HCl solution to a target pH of 6.5 (range 6.0-7.0) and stirred until a clear solution is obtained. Slowly add 1 g of peptoid and stir until completely dissolved for a target concentration of 1 mg/mL (0.1% w/v). This solution can be sterilized by using a 0.22 μm filter and stored in a sterile container with closure until use.

Additional example peptoid formulations are described in Example 5.

The peptoid may be included in the formulation over a range of 0.005%-5%. Alternate buffer agents include histidine buffer for pH control in the physiological range, and may be utilized over a molarity range of 10 mM-100 mM. Preservatives may be included in the range of 0.05%-2%, and can also include, but are not limited to, benzalkonium chloride and sodium benzoate.

In some embodiments, the compositions described herein may be formulated as mixtures of one or more peptoids. For example, these mixtures may comprise peptoids in various molar ratios, such as 0.01:0.99 to 0.99:0.01, or any ratio in between. In some embodiments, the effective amount may be from 1-1000 mg/day, with a preferred embodiment of 25-750 mg/day, or a more preferred embodiment of 50-500 mg/day, or an even more preferred embodiment of 100-400 mg/day.

In some embodiments, a composition may comprise a peptoid compound described herein in mixtures or combinations with other antiviral agents, such as known antiviral compounds. In some embodiments, the peptoid compounds of the present disclosure may act synergistically with the known antiviral compounds, so that the resulting composition demonstrates improved effectiveness.

In some embodiments, the present disclosure relates to a method of inhibiting or preventing replication of a virus of the Filoviridae family.

In some embodiments, one or more peptoid compounds described herein can be used in compositions for application to a surface of an object, or for use as a cleaning agent, for cleaning an object, an/or as a cleaning solution for a subject's skin, to prevent or treat an infection of a virus of the Filoviridae family.

In some embodiments, one or more peptoids compounds suitable for use as agent effective to inhibit or prevent replication of a virus of the Filoviridae family on the surface of an object or on the surface of a subject's skin may include peptoid compound MXB-27,369, peptoid compound MXB-25,739, peptoid compound MXB-24,656, peptoid compound MXB-24,816, or any combination thereof.

In some embodiments, the method comprises contacting the virus with one or more peptoid compounds selected from: a peptoid compound MXB-27,369 having a formula H-Ndec-Nlys-Nspe-Nspe(p-Br)-Nlys-Npse-Nspe(p-Br)-NH₂, a peptoid compound MXB-25,739 having a formula H-Ndec-(Nlys-Nspe-Nspe (p-Br))₂-Nlys-NH₂, a peptoid compound MXB-24,656 having a formula H-Nlys-Nspe(p-Br)-Nspe(p-Br)-Nlys-Nspe(p-Br)₅-Nspe(p-Br)-NH₂, a peptoid compound MXB-24,816 having a formula H-Ntetradec-Nlys-Nspe-Nspe-Nlys-NH₂, or any combination thereof, in an amount and for a time sufficient to inhibit or prevent replication of the virus. In some embodiments, the replication of the virus may be decreased by up to 50%, 60%, 70%, 80%, 90%, 95% or 100%. In some embodiments, the amount of peptoid sufficient to inhibit or prevent replication of the virus may be from 1-1000 mg/day, with a preferred embodiment of 25-750 mg/day, or a more preferred embodiment of 50-500 mg/day, or an even more preferred embodiment of 100-400 mg/day.

In some embodiments, the virus is Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.

In some embodiments, the method of inhibiting or preventing replication of a virus of the Filoviridae family may include binding the peptoid to a surface of an object, thereby conferring antiviral activity to the object. Accordingly, in some embodiments, binding the peptoid to a surface of an object may prevent or inhibit an infection of a virus of the Filoviridae family in a subject through contacting with the object.

In some embodiments, the methods described herein may include binding or incorporating the peptoids described herein into substrates to confer antiviral activity to an object.

The peptoid compounds of the present disclosure and compositions thereof may be bound to or incorporated into substrates to provide antiviral substrates to reduce or inhibit viral contamination of the substrate. The present disclosure also provides objects comprising the antiviral substrates of the invention.

Accordingly, in some embodiments, a composition comprising one or more peptoids may be applied to an object, for example applied to a surface of an object. Applying a composition described herein may provide antiviral activity to the object according to the present disclosure.

In some embodiments, a composition comprising one or more peptoids can be used as an antiviral cleaning solution that is suitable for cleaning an object.

In some embodiments, the object may be comprised of ceramics, polymers, glass, metal, metal oxides, and composites comprised of ceramics, glass, metal or metal oxides plus polymers as described above. Suitable metals include steel, stainless steel, aluminum, copper, titanium, alloys thereof, and combinations thereof. Objects may be in the form of or comprise an extrudate, film, membrane, laminate, knit fabric, woven fabric, nonwoven fabric, fiber, filament, yarn, pellet, coating, or foam. Objects may be prepared by any means known in the art, such as, but not limited to, methods of injection molding, extruding, blow molding, thermoforming, solution casting, film blowing, knitting, weaving, or spinning. Objects may be items of apparel, medical garments or barrier materials, such as gowns, masks, gloves, slippers, booties, head coverings or drapes, among others. Objects may be medical materials, tools, devices, or implants, among others. The substrate may be used in bedding, bed linens, cleaning wipes, shower curtains, towels, washcloths, mops, table cloths, walls, and counter surfaces, among others.

In some embodiments, for example and not by way of limitation, the substrates may include polymers selected from the group consisting of latex, polyvinyl chloride, polyimide, polyesters, polyethylene, polypropylene, polyamides, polyacrylates, polyolefins, polysaccharides, polyurethane, polysulfone, polyethersulfone, polycarbonate, fluoropolymers, cellulosics, synthetic rubber, silk, silicone, and mixtures or blends thereof. Additional polymer substrates are also functionalized polymer substrates comprising the aforementioned polymers, which may further comprise or may be functionalized to comprise active groups with which peptoid oligomers may react, and which allow for immobilization of same. Examples of active groups include, but are not limited to: acrylic acid, acetal, hydroxyl, amines, epoxides, carboxylates, anhydrides, isocyanates, thioisocyanates, azides, aldehydes, halides, acyl halides, aryl halides and ketones at 1 to 50% by weight of the polymer. Various methods of protein or peptide immobilization are described in Protein Immobilization (Richard F. Taylor (ed.), Marcel Dekker, New York, 1991); similar methods may be used such as those familiar to practitioners skilled in the art of immobilization of peptoids.

In some embodiments, the substrates may include ceramics, polymers, glass, metal, metal oxides, and composites comprised of ceramics, glass, metal or metal oxides plus polymers as described above. Suitable metals include steel, stainless steel, aluminum, copper, titanium, alloys thereof, and combinations thereof.

Peptoid compounds can be bound to a substrate physicochemically or covalently. Physicochemical binding of peptoids to the substrate may occur by any one or combinations of the following forces: electrostatic, hydrogen bonding, and Van der Waals. Alternatively, peptoids may be bound to the substrate surface by a covalent bond. Additionally, peptoids of the present disclosure can be incorporated into a polymer by mixing with the polymer, for example by dissolving the peptoid and the polymer in a common solvent and casting or molding the peptoid: polymer mixture into an object.

In some embodiments, a peptoid may be bound to the substrate by coating a substrate polymer with an aqueous or non-aqueous solution of the peptoid, wherein the peptoid is at concentration ranging from about 0.0001 to about 20 weight percent. The peptoid is contacted with the substrate polymer, and the peptoid and substrate polymer are optionally shaken at temperatures ranging from about −10° C. to about 150° C. for a period of time ranging from about 0.1 min to about 96 hrs. The peptoid and substrate polymer may be shaken at a temperature of from about 25° C. to about 80° C. for a period of time ranging from about 1 min to about 24 hrs.

In some embodiments, the substrate polymer may be primed to generate active groups that will bind to the peptoid. Surface modification of the polymer may be achieved by a variety of techniques well known in the art including: oxidation, reduction, hydrolysis, plasma, and irradiation. Substrate polymers containing acid or base hydrolysable groups such as polyesters, polyamides, and polyurethanes may be treated with acid or base first. Subsequently, the hydrolyzed polymer is brought into contact with an aqueous or non-aqueous solution of from about 0.001 to about 20 weight percent of the peptoid. The peptoid and the polymer may be shaken at temperatures ranging from about −10° C. to about 150° C. for a period of time ranging from about 0.1 min to about 96 hrs. The peptoid and substrate polymer may be shaken at a temperature of from about 25° C. to about 80° C. for a period of time ranging from about 10 min to about 24 hrs.

In some embodiments, a substrate polymer containing 1-50% active groups is brought into contact with an aqueous or non-aqueous solution comprising from about 0.0001 to about 20 weight percent of the peptoid.

Methods for binding or incorporating peptides and/or peptoids to substrates are known to those of skill in the art. Additional modifications to the above general guidelines can be implemented, as required, to improve binding or incorporation of the peptoids to substrates. U.S. Pat. No. 7,307,061, for example, describes such methods in detail and is incorporated herein in its entirety.

After treatment with the peptoid, the object may be washed, for example with deionized water. Optionally, the object may then be dried via methods known in the art. Such methods include ambient air drying, oven drying, and air forced drying. In some embodiments, the object may be dried at about 50° C. to about 120° C., or at about 50° C. to about 100° C., for about 15 min to about 24 hrs.

Objects comprising the substrate may be in the form of or comprise an extrudate, film, membrane, laminate, knit fabric, woven fabric, nonwoven fabric, fiber, filament, yarn, pellet, coating, or foam. Objects may be prepared by any means known in the art, such as, but not limited to, methods of injection molding, extruding, blow molding, thermoforming, solution casting, film blowing, knitting, weaving, or spinning. In some embodiments, metal objects may be coated directly, or a coating of a polymer or functionalized polymer may first be applied to the metal surface. Alternatively, a film of such a polymer or functionalized polymer may be coated with a peptoid and then applied to the metal surface.

The substrate may be used In items of apparel, medical garments or barrier materials, such as gowns, masks, gloves, slippers, booties, head coverings or drapes, among others. The substrate may be used in medical materials, tools, devices, or implants, among others. The substrate may be used in bedding, bed linens, cleaning wipes, shower curtains, towels, washcloths, mops, table cloths, walls, and counter surfaces, among others.

Other methods of binding the antiviral peptoids described herein to objects are described in International Application Publication No.'s WO2023287570A2 and WO2022120393A2, the disclosures of which are incorporated herein by reference in their entireties.

In some embodiments, a composition comprising one or more peptoids can be used as an antiviral cleaning solution that is suitable for applying to skin of a subject, for example without limitation as a hand wash or body wash.

Accordingly, in some embodiments, the one or more peptoids may be formulated for topical use as a cleaning solution.

In some embodiments, the present disclosure relates to methods of applying a composition comprising the peptoid as a cleaning solution to an object, or to subject's skin.

### EXAMPLES

The present examples are provided for illustrative purposes only. They are not intended to and should not be interpreted to encompass the full breadth of the invention.

### Example 1. Preparation of Peptoid Compounds

The peptoid compounds listed in Table 1 were prepared using a sub-monomer protocol, on Rink Amide MBHA Resin. Example sub-monomer protocols are described in Zuckermann, R. N., Kerr, J. M., Kent, S. B. H., & Moos, W. H. (1992) J. Am. Chem. Soc., 114, 10646-10647 and in U.S. Pat. Nos. 8,445,632 and 6,887,845, the entireties of which are incorporated herein by reference. The starting reagents are bromoacetic acid and a small set of primary amines that are readily available commercially. The crude peptoid products were then cleaved from the resin and sidechain protective groups were removed in one step by acidolysis. The resulting residue was then resolubilized and lyophilized twice to produce peptoids as a dry powder. The peptoid products were then purified by HPLC to produce peptoids in powder form, with hydrochloride as the counter ion. Peptoid compounds were stored as dry powder at −20° C. and protected from light prior to preparation of stock solutions.

TABLE 1
Peptoid compounds tested against Ebola virus

| Peptoid | | Molecular | | |
|---|---|---|---|---|
| compound | | weight without | Molecular | |
| name | Peptoid Sequence | HCl salt (g/mol) | Formula | Counter Ion |
| MXB-24,656 | H-Nlys-Nspe(p-Br)- | 1233.78 | C₅₂H₆₇Br₄N₉O₆ | Hydrochloride |
| | Nspe(p-Br)-Nlys- | (1343.15 with | | |
| | Nspe(p-Br)₅-Nspe(p- | HCl salt) | | |
| | Br)-NH₂ | | | |
| MXB-22,510 | H-Ntridec-Nlys-Nspe- | 835.19 | C₄₇H₇₈N₈O₅ | Hydrochloride |
| | Nspe-Nlys- NH₂ | (944.56 with | | |
| | | HCl salt) | | |
| MXB-27,369 | H-Ndec-Nlys-Nspe- | 1273.31 | C₆₄H₉₂Br₂N₁₀O₇ | Hydrochloride |
| | Nspe(p-Br)-Nlys-Npse- | (1382.68 with | | |
| | Nspe(p-Br)-NH₂ | HCl salt) | | |
| MXB-25,605 | H-Ndodec-Nlys-Nspe- | 821.17 | C₄₆H₇₆N₈O₅ | Hydrochloride |
| | Nspe-Nlys-NH₂ | (930.53 with | | |
| | | HCl salt) | | |
| MXB-24,816 | H-Ntetradec-Nlys-Nspe- | 849.22 | C₄₈H₈₀N₈O₅ | Hydrochloride |
| | Nspe-Nlys-NH₂ | (958.58 with | | |
| | | HCl salt) | | |
| MXB-25,739 | H-Ndec-Nlys-Nspe- | 1401.49 | C₇₀H₁₀₄Br₂N₁₂O₈ | Hydrochloride |
| | Nspe(p-Br)-Nlys-Nspe- | (1547.32 with | | |
| | Nspe(p-Br)-Nlys-NH₂ | HCl salt) | | |
| MXB-21,008 | H-(Nbz-Nme)3-NH2 | 803.96 | C₄₂H₅₇N₇O₉ | — |

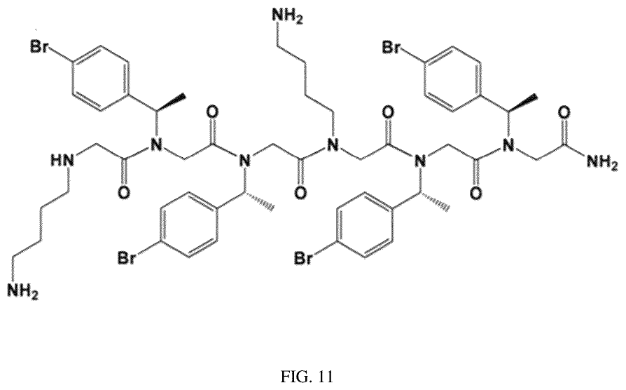

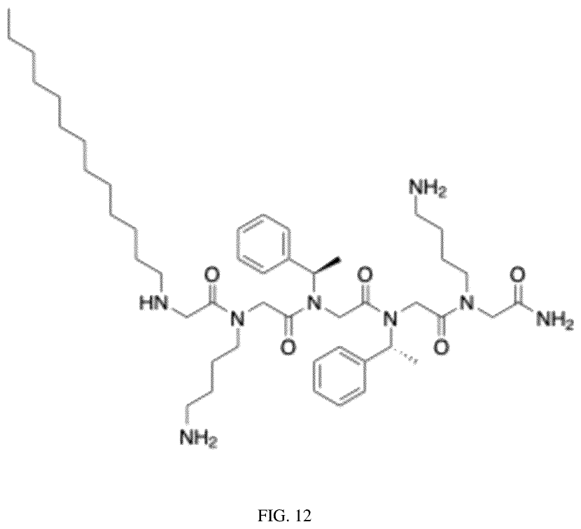

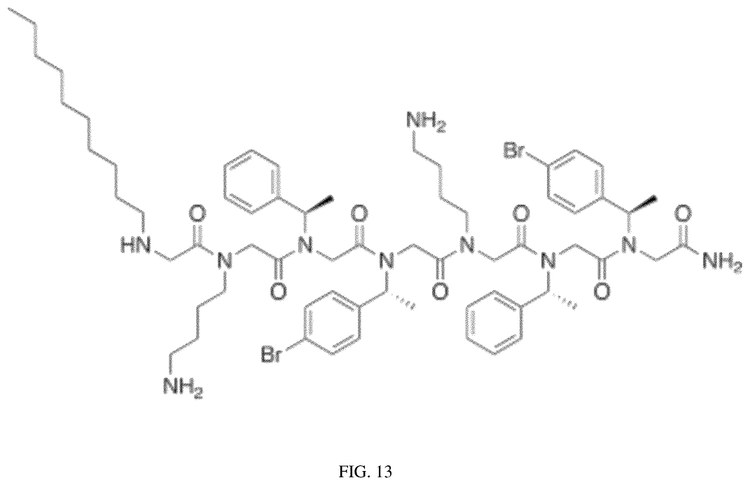

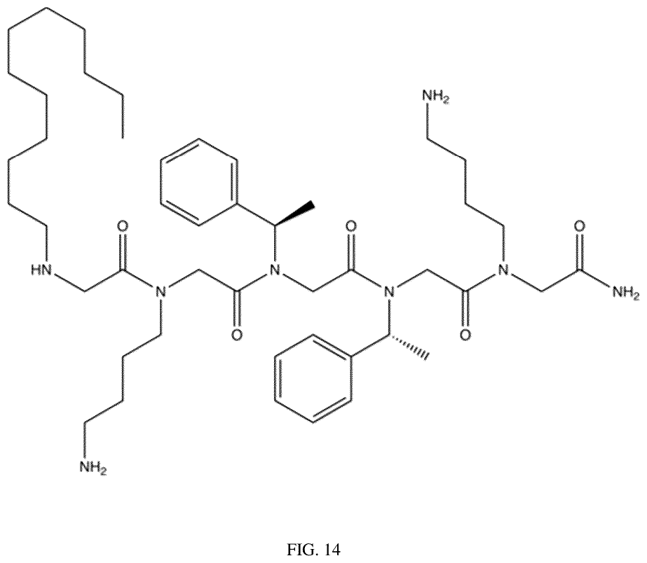

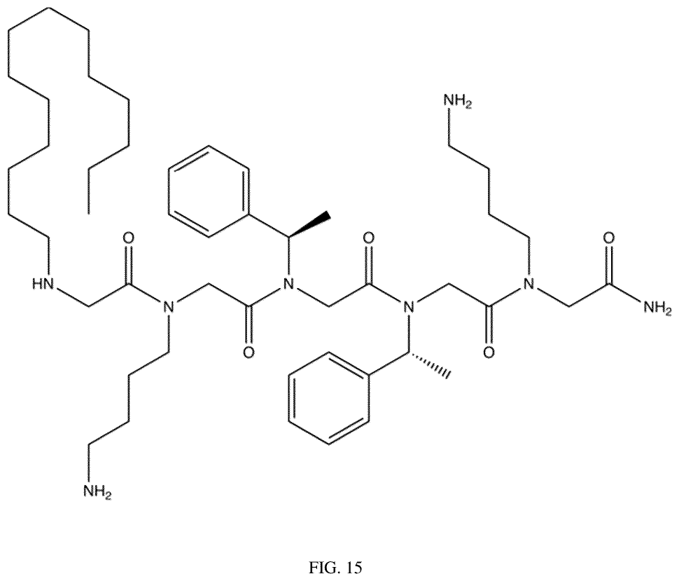

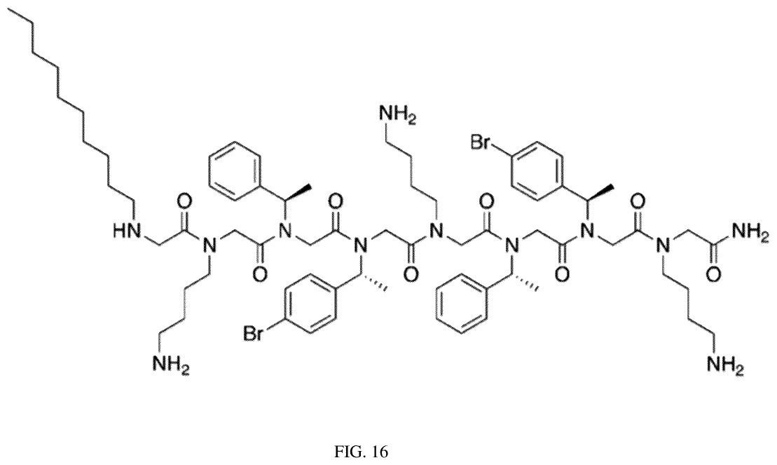

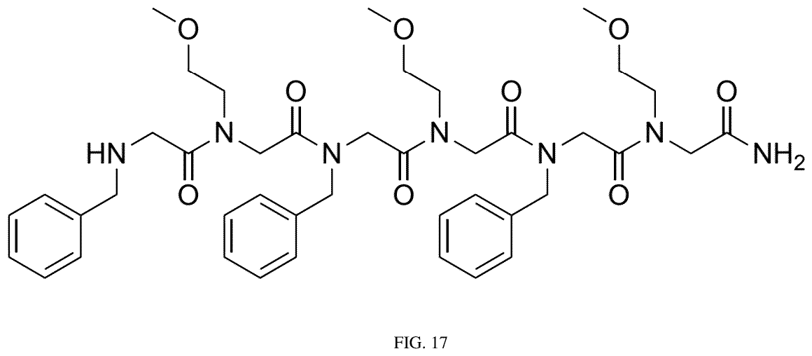

Chemical structures of the peptoid compounds listed in Table 1 are shown in FIG. 11-FIG. 17.

An initial stock concentration of each peptoid compound was prepared in tubes at 2 mg/ml in phosphate-buffered saline (PBS) pH 7.4 (Gibco; cat no. 10010023). Initial dissolution of lyophilized peptoid compound powders to create a stock solution was performed by gentle mixing by inverting the stock solution tube several times), followed by checking for turbidity, precipitation, or aggregate before proceeding to the next steps. If gentle inversion was insufficient to achieve a solution, the stock solution tube was briefly vortexed. The stock solution was then checked for any undissolved particulate, aggregates, or precipitation before proceeding to the next step. If gentle inversion and vortexing was insufficient to achieve a solution, the stock solution tube was briefly sonicated for 15-60 seconds. The stock solution was then checked again for any undissolved particulate, aggregates, or precipitation before proceeding to the next step. If turbidity, precipitation, or aggregate was observed at the initial stock concentration, the initial stock concentration was solubilized by diluting further in PBS to 1 mg/ml. Aliquots of the stock solutions were dispensed in polypropylene vials, protected from light, and stored at −20° C. or −80° C. prior to use.

Before testing, the aliquots were carefully observed for any signs of turbidity, precipitation or aggregate during sample preparation and were mixed, vortexed, or sonicated as needed.

### Example 2. Testing In Vitro Activity of Peptoid Compounds against Ebola Virus Replication

Each of the peptoid compounds listed in Table 1 were tested against a transgenic Zaire Ebola virus (EBOV) of the Mayinga strain, expressing green fluorescent protein (EBOV-GFP) (Ebihara et al. (2007) The Journal of Infectious Diseases 196:S313-22), as follows:
1. VeroE6 cells were seeded 1:3 in a 24-well plate in Dulbecco's Modified Eagle's Medium (DMEM)/10% fetal bovine serum (FBS).
2. Stock solutions of peptoid compounds were prepared as described in Example 1 at 2 mg/ml in PBS pH 7.4.
3. Peptoid compound stock solutions were diluted in 200 μL of DMEM at concentrations of 40 μg/mL and 200 μg/mL. The Ebola virus stock was prepared as 2× concentration suspended in 200 ul of DMEM. 180 μL of each peptoid compound stock solution was mixed with and 180 μL of the virus suspension. The final concentrations of the peptoid compounds were 20 μg/mL and 100 μg/mL. The virus-peptoid compound mixtures were incubated for two hours at 37° C.
4. After incubation at 37° C. for 2 hour, 100 μL of the virus-peptoid compound mixture was added to the VeroE6 cells cultured in 24-well plates. All treatments were performed in triplicate.
5. The treated cells were cultured for another 1 hour at 37° C. in a 5% CO₂ incubator.
6. The 23-well plates were washed 3 times with 0.5 mL DMEM per well after infection and replenished with DMEM/2% FBS (1 mL per well).
7. At time points 0, 24, 48, 72, and 96 hours post-infection, 50 μL cell culture media was removed from each well and stored at −80° C. until titration.
8. Fluorescence microscopy was used to visualize the GFP-positive virus particles present in each well of the cultured VeroE6 cells in each treatment group, at each time point.
9. Viral titers in the samples were determined by the median Tissue Culture Infectious Dose (TCID₅₀) and expressed as log₁₀TCID₅₀/mL. Antiviral potency of each peptoid compound was measured by its inhibitory effects of virus replication in cell culture in comparing treated versus untreated cells.

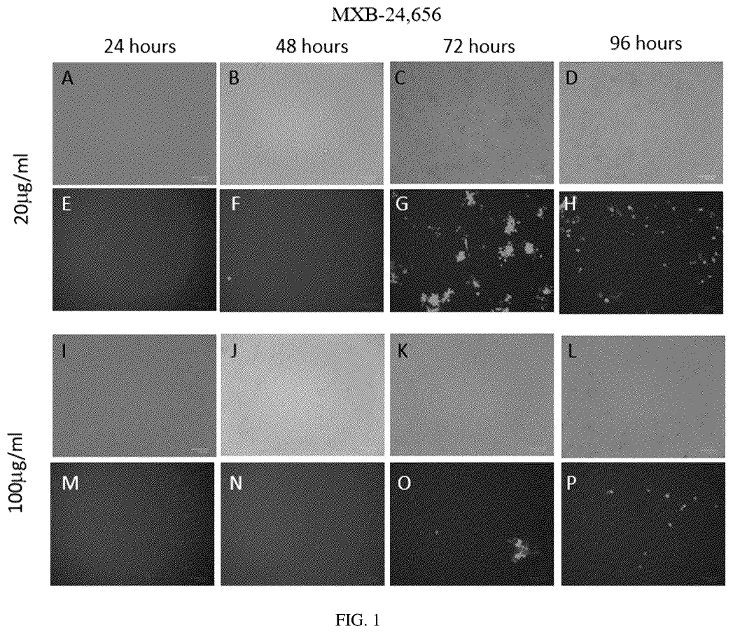

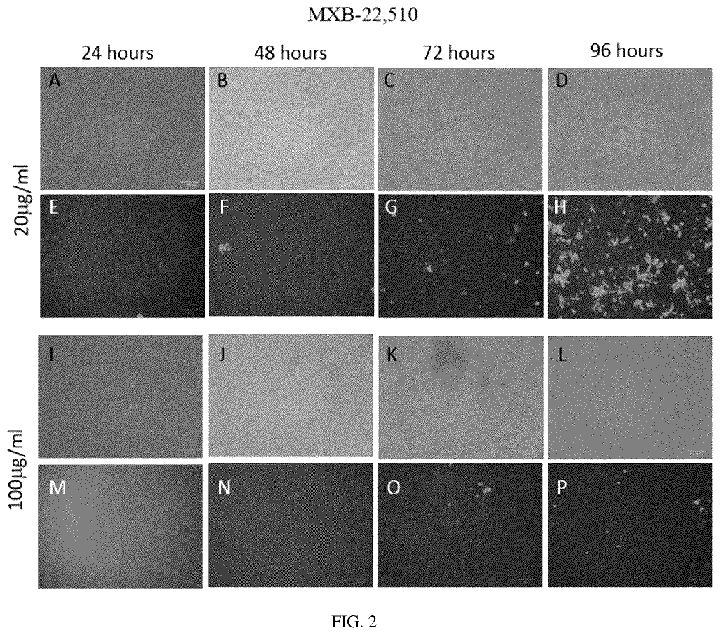

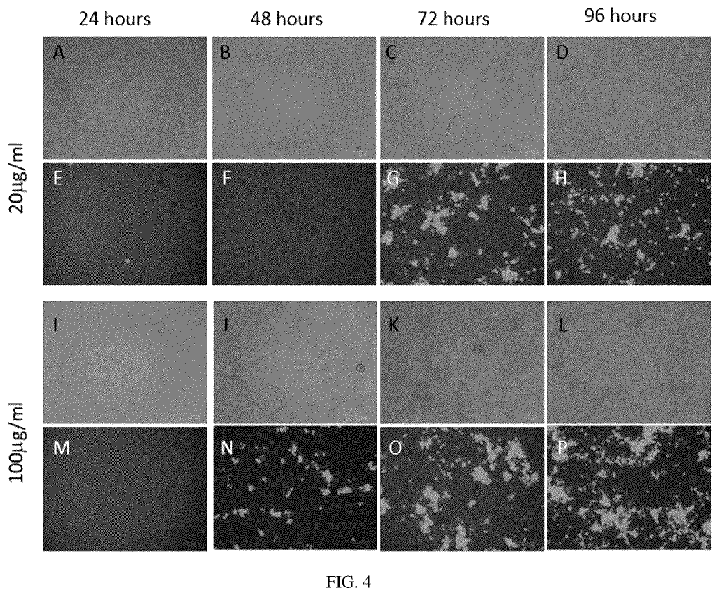

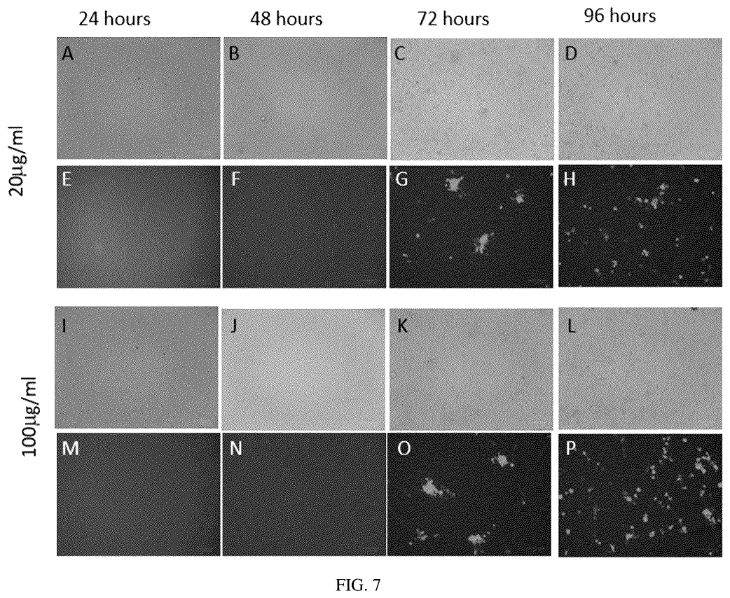

Example micrographs of the treated VeroE6 cells are shown in FIG. 1-8. For each of FIG. 1-8, micrograph images labeled E, F, G, and H respectively show example GFP fluorescence at 24 hours, 48 hours, 72 hours, and 96 hours after the indicated treatment. Similarly, for each of FIG. 1-8, micrograph images labeled M, N, O, and P respectively show example GFP fluorescence at 24 hours, 48 hours, 72 hours, and 96 hours after the indicated treatment.

For each of FIG. 1-8 micrograph images labeled A, B, C, and D respectively are example brightfield micrograph images at 24 hours, 48 hours, 72 hours, and 96 hours after the indicated treatment.

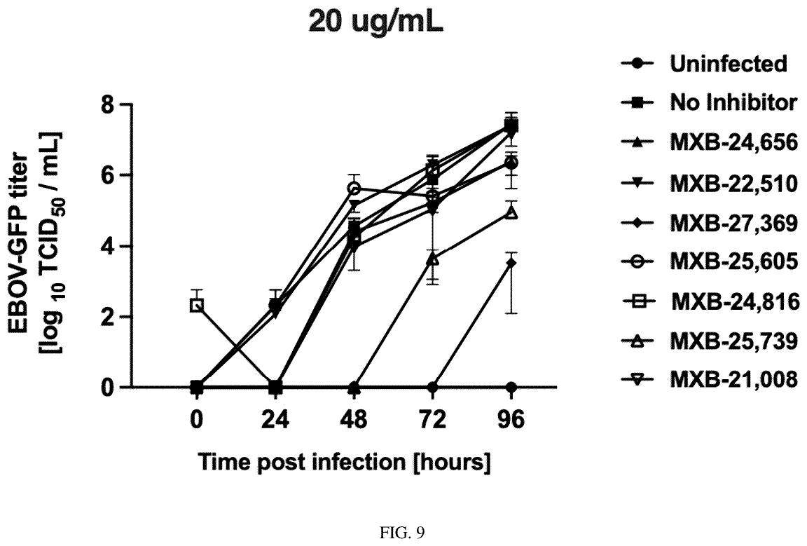

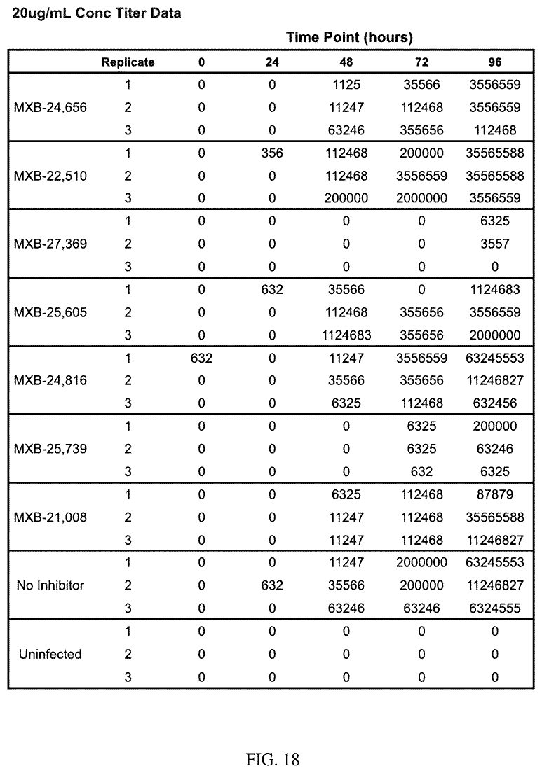

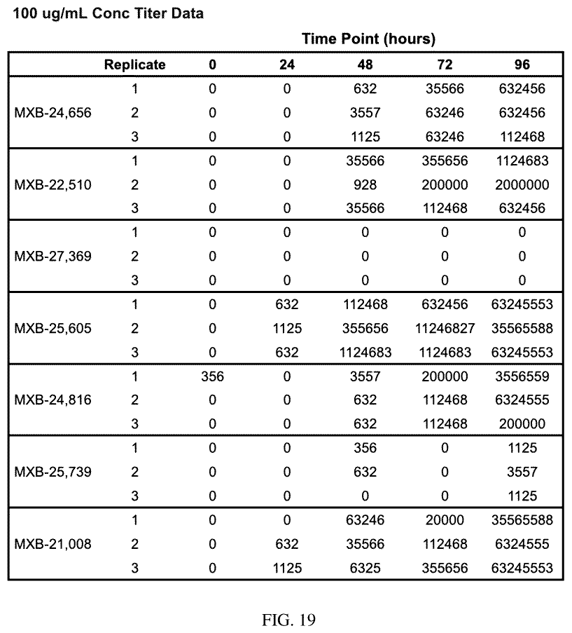

FIG. 9-FIG. 10 and FIG. 18-19, respectively, are graphs and Tables reporting viral titers of samples determined by the TCID50 and expressed as LogTCID50/ml. Antiviral potency was measured by its inhibitory effects of virus replication in cell culture in comparing treated versus untreated cells.

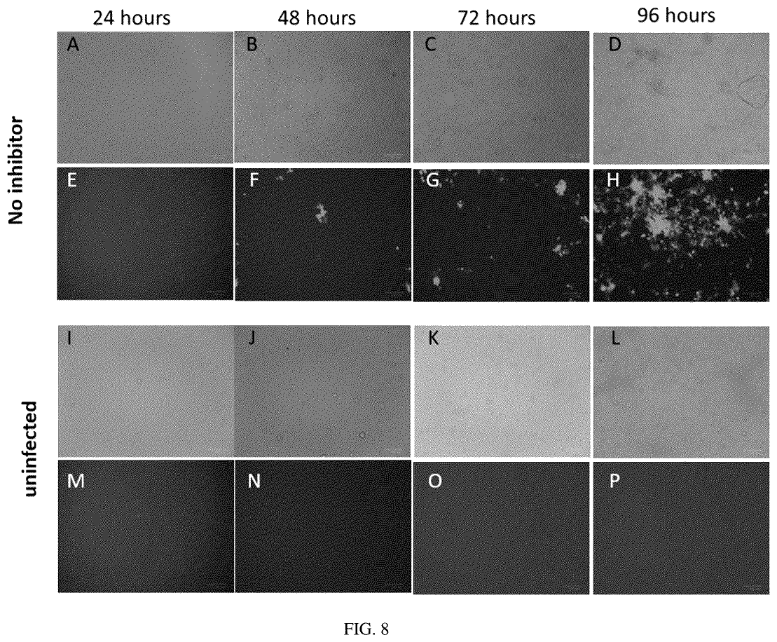

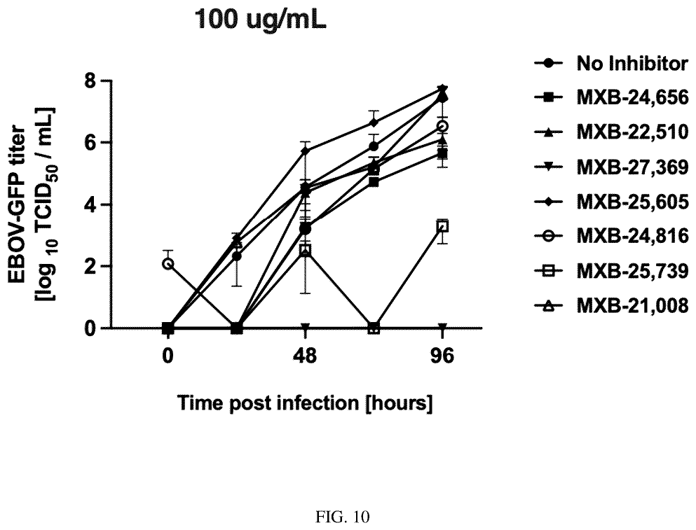

Uninfected VeroE6 cells showed no viral titer (FIG. 9) and no GFP fluorescence signal at any time point (FIG. 8, M-P). In contrast, VeroE6 cells infected with EBOV-GFP that had not been incubated with a peptoid compound (“No inhibitor”) showed viral titers (FIG. 9 and FIG. 10) and GFP fluorescence reporter expression (FIG. 8, E-H) that increased from 24 hours to 96 hours post-infection.

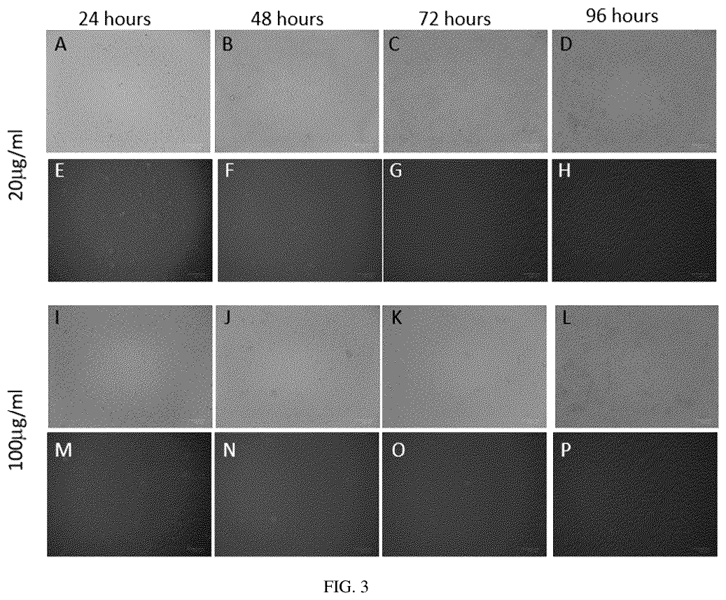

Peptoid compound MXB-27,369 showed robust inhibition of viral titers and GFP-positive Ebola virus replication in VeroE6 cells at both doses tested of 20 μg/mL and 100 μg/mL (FIG. 3, FIG. 9 and FIG. 10). For peptoid compound MXB-27,369, log₁₀TCID₅₀/mL values of zero were observed at all time points when tested in VeroE6 cells at a dose of 100 μg/mL. When tested at 20 μg/mL, peptoid compound MXB-27,369 resulted in log₁₀TCID₅₀/mL values of zero up to 72 hours post-infection, and also resulted in log₁₀TCID₅₀/mL values lower than the other peptoid compounds tested at the last time point tested at 96 hours post-infection.

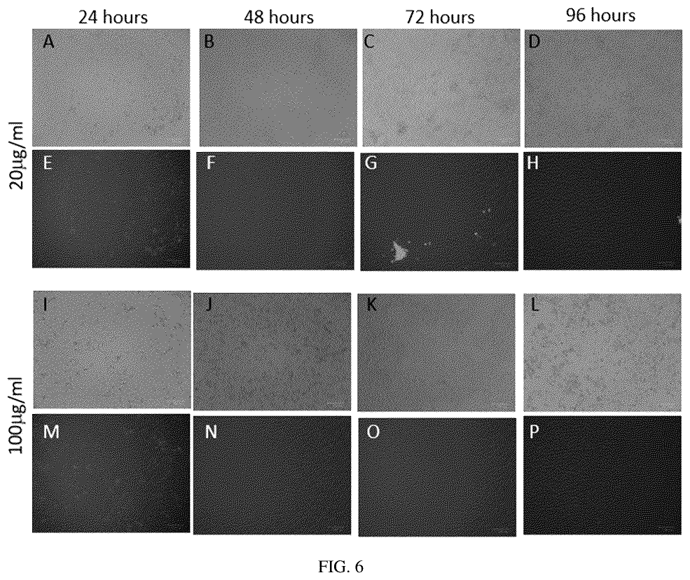

Peptoid compound MXB-25,379 also showed antiviral activity against Ebola virus following incubation at 20 μg/mL and 100 μg/mL doses (FIG. 6, FIG. 9 and FIG. 10).

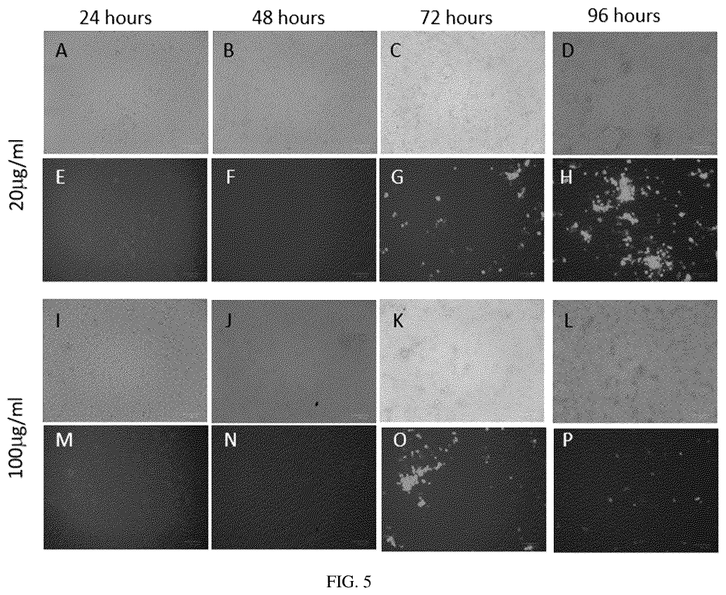

Peptoid compounds MXB-24,656 (FIG. 1 and FIG. 10), and MXB-24,816 (FIG. 5 and FIG. 10) showed antiviral activity against Ebola virus following incubation at 100 μg/mL with Ebola virus.

None of the other peptoid compounds tested showed antiviral activity against GFP-positive Ebola virus replication in VeroE6 cells (FIG. 9 and FIG. 10).

### Prophetic Example 3. Testing In Vivo Activity of Peptoid Compounds against Ebola Virus in Mice

This example describes a prophetic animal study in which the peptoid compounds listed in Table 1 are tested against EBOV-GFP in mice.

An in vivo model of Ebola virus infection in mice may be established following experimental protocols as described in Ebihara et al. (2007) The Journal of Infectious Diseases 196:S313-22, incorporated by reference herein, for example as follows.

To assay the virulence of EBOV-GFP in mice without peptoid compound treatment, groups of 3-6 mice may be injected intraperitoneally, at 2 different sites (100 mL per site), with virus dilutions ranging from 10⁴ to 2×10⁻¹ ffu/mL. After infection, mice may be scored for clinical signs, and weight loss recorded for a minimum of 11 days after infection. For all surviving animals, observation may be continued until day 21 after infection. To assess disease progression based on GFP expression, groups of 9 mice may be infected intraperitoneally with 1000 ffu of EBOV-GFP. Spleen and liver samples may be collected from 1 uninfected and 4 infected mice on days 3 and 5 after infection. The collected organ samples may be homogenized and fixed in 4% PFA. The tissue samples may be analyzed on a LSRII flow cytometer with FACSDiva software.

To assay antiviral activity of peptoid compounds listed in Table 1 are tested against EBOV-GFP in mice, groups of mice infected with EBOV-GFP as above may be administered with each of the peptoid compounds listed in Table 1. Dosage of peptoid compounds may be administered subcutaneously to mice at doses of 0.1-20 mg/kg in phosphate-buffered saline

It is expected that in vivo results will be similar to the in vitro results described in Example 2, such that peptoid compounds MXB-27,369 and MXB-25,739 may show antiviral activity at at least one dose, showing inhibition of GFP-positive Ebola virus replication in mice, and improved survival and health of the infected mice.

### Example 4. Example Peptoids Safety Data

A cytotoxicity study of peptoids was conducted using the EpiOral air-liquid interface system (a three-dimensional mucociliary tissue model consisting of primary cultures normal human-derived oral epithelial cells (EpiOral, MatTek Life Sciences, Ashland, MA, USA), Peptoids were incubated with the cells at 2-fold dilutions from 256 μg/mL to 32 μg/mL for 3 hours at 37° C. At the end of the incubation, the supernatant was aspirated, cultures were rinsed, and 3-(4,5-dimethylthylthiazol-2-y) (MTT) was added to assess cell viability. The extent of cell viability was assessed as the optical density of extracted samples measured at 570 nm. The results showed no observable cytotoxicity of peptoids as assessed by this metabolic assay, at concentrations up to 256 μg/mL.

A bacterial reverse mutation assay was conducted with peptoids using the plate incorporation method and bacterial strains Salmonella typhimurium TA98, TA100, TA1535, and TA1537 and Escherichia coli (E. coli) WP2uvrA in both the presence and absence of liver S9 obtained from rats induced with phenobarbital/β-naphthaflavone. Peptoids were evaluated for cytotoxicity at 0.005, 0.05, 5, 16, 50, 160, 500, 1600, and 5000 μg/plate and was found to produce >50% toxicity. Concentrations of peptoid showing acceptable bacterial survival produced no increase in revertant colonies exceeding that observed in DMSO controls by more than two-fold, Therefore, peptoids were considered to be non mutagenic under the conditions of this assay.

The ability of peptoids to cause the release of cytokines from human peripheral blood mononuclear cells (PBMCs) was evaluated. In this assay, PBMCs from a single donor were plated at a density of 2×10⁵ cells/well and incubated with Maxwell peptoids (20, 40, or 100 μg/mL) for 48 hours at 37° C. after which supernatants were collected and analyzed for the presence of a panel of cytokines (IFNγ, IL-1β, IL-2, IL-6, IL-8, IL-10, IL-17A, MIP-1α, TNFα), Anti-CD3 (15 μg/mL), superagonist anti-CD28 (15 μg/mL), lipopolysaccharide (100 ng/ml), and phytohemagglutinin (10 μg/mL) were used as positive controls. Peptoids did not cause the release of any of the pro-inflammatory cytokines evaluated.

Peptoids were evaluated for the potential for local adverse effects in nasal tissues using the MucilAir-Pool™ in vitro assay and repeated daily dosing for 4 days. No significant decrease in tissue integrity as measured by trans-epithelial electrical resistance (measured on Days 1-4) was observed. In addition, no decrease in mucociliary clearance frequency (measured on Day 4) or increases in cytotoxicity as measured by lactate dehydrogenase (measured on Days 2 and 4) were observed. A small decrease in ciliary beat frequency was observed on Days 2 and 4. Increases in inflammation-induced RANTES (Day 4) and GM-CSF (Day 2) release into the culture medium were also observed, but only at the highest concentration tested (100 μM). Further, no effects on several inflammatory cytokines (IL-8, IL-6, or β-defensin2; measured on Days 2 and 4) released into the culture medium were observed.

The toxicity and toxicokinetics associated with 7 days of repeated intranasal dosing of Sprague-Dawley rats with peptoids was evaluated. Rats were administered peptoid at a volume of 25 μL per nostril with both nostrils treated twice daily (i.e., total dose volume=50 μL/nostril or 100 μL/day) for total daily doses of 0 (vehicle; phosphate-buffered saline), 0.625, 2.5, and 10 mg/rat. All animals survived to their designated days of sacrifice. Potentially treatment-related clinical signs were limited to an increase in abnormal respiratory sounds. This sign was also noted in two vehicle control rats, as well as in five low dose (4M/1F), 8 middle dose (5M/3M), and 10 (5M/5F) high dose of the peptoid treated rats. There were no significant effects on rat body weights, but males at the high dose had body weights that were 11% less than the controls at necropsy. No treatment-related effects were observed at necropsy and no statistically significant effects on organ weights were detected.

### Example 5. Example Formulations of Peptoid Compounds

The peptoids described herein may be formulated into a wide variety of dosage forms for topical, targeted local delivery, or systemic delivery as may be required by the type of wound.

### Topical Formulation Approaches

Topical formulation approaches include powder, solution, suspension, semisolids, or infused into a bandage or other dressing material. Powder formulations include, but are not limited to, powder, granulation, pellets, or mini tablets. These powder dosage forms may be packaged or contained in a simple stick pack, sachet, vial, spray, shaker bottle, or multi-use bottle. The solution formulations may be provided as a solution, granules or powder for reconstitution, disintegrating tablet for dissolution and reconstitution, or incorporated into a spray bottle, with or without materials to provide a scaffold or topical bandage to protect the wound. The suspension formulations include, but are not limited to, aqueous suspension, suspension in another solvent, granules or powder for suspension, or disintegrating tablet for resuspension. These suspension dosage forms may be packaged or contained in a simple stick pack, sachet, vial, shaker bottle, or multi-use bottle. Semisolid formulation approaches include, but are not limited to, I, gel, ointment, lotion, paste, balm, salve, emulsion, suppository (e.g. embedded in wax or polymer that liquifies at body temperature), spray, including spray on bandages, foam, including spray on foams, or film. Peptoids can also be infused into medical wound dressings including gauze, bandages, and wound packing, among others. Peptoids may be packed into a wound using a disintegrating tablet, drug eluting tablet or tablets, drug eluting beads or granules, or implantable, self dissolving sheet, wafer, block or suppository (e.g. embedded in wax or polymer that liquifies at body temperature), or thin wafer inserted for drug elution at the local wound site.

### Other Delivery Methods

In addition to topical formulation approaches, alternate administration approaches of the peptoids may be employed. These approaches include injection for local delivery, injection for systemic delivery, transdermal patch or other transdermal approach, intravenous, intranasal, intraocular, aural delivery, sublingual, buccal, or oral delivery, including immediate-release dosage forms, as well as modified release. Injection delivery methods may include intraperitoneal, subcutaneous, intramuscular, intrathecal, or intravenous.

### Example Powder Formulation

The peptoids may be administered as a simple powder as a standalone drug or with additional excipients to improve flowability or other processing requirements. This powder may be filled into a hard gelatin capsule and subsequently filled into bottles, or packaged in a sachet, stick pack, vial, or other container to aid in portability and ease of administration. This powder formulation may be applied directly to the wound, or dissolved in an aqueous vehicle for topical administration. A powder for topical administration may be prepared using the following formula:

### Formulation 1

Drug Substance—600 g (active ingredient)
Microcrystalline cellulose—100 g (processing aid-flowability)
Lactose—300 g (processing aid-flowability)
   Step 1—blend the microcrystalline cellulose and lactose in a suitable blender and blend for 10 minutes
   Step 2—add the drug substance and blend for an additional 10 minutes, or until the drug is uniformly distributed throughout the blender.
   Step 3—discharge the powder blend from the blender into a suitable bin or container to store until the filling operation.
   Step 4—fill the appropriate amount of blend into each package for storage, transfer, and administration.

### Example 2—Granulation

The drug substance powder can also be incorporated into a granulation that can produce a particle with improved flowability and density relative to the powder in Example 1. This granulation can be prepared either dry, or in the presence of water or other solvent. The binder may be added either wet (in the granulation solution) or dry with the rest of the materials. If water or other solvent is used, the blend is dried in a suitable pharmaceutical drier, such as a vacuum oven, forced air oven, or fluid bed drier. This granulation may be filled into a hard gelatin capsule and subsequently filled into bottles, or packaged in a sachet, stick pack, vial, or other container to aid in portability and ease of administration. This granulation formulation may be applied directly to the wound, or dissolved in an aqueous vehicle for topical administration. A granule formulation that may be used for topical application or dissolution into a topical solution may be prepared using the following formula:

### Formulation 2

Drug substance—700 g (active ingredient)
Microcrystalline cellulose—200 g (diluent and processing aid)
Povidone—100 g (binder)
Water (processing aid; removed during processing)
   Step 1—add the active and excipients to a suitable pharmaceutical mixer or granulator such as a planetary mixer, high-shear granulator, fluid bed granulator, or extruder.
   Step 2—slowly add the water while the mixer is operating until all the water has been added.
   Step 3—continue the granulation step until the granulation endpoint is achieved.
   Step 4—discharge the wet mass into a container suitable to hold the material until drying.
   Step 5—charge the wet mass into a suitable drier and dry until the endpoint of less than 2% water is reached.
   Step 6—discharge the dried granulation into a suitable bin or container to store until the filling operation.
   Step 7—fill the appropriate amount of granulation into each package for storage, transfer, and administration.
      Additional formulation examples are shown below, and follow a similar procedure for preparation.

### Formulation 3

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 4

Drug substance—600 g (active ingredient)
Microcrystalline cellulose—300 g (diluent and processing aid)
Hydroxypropyl cellulose—100 g (binder)
Water (processing aid; removed during processing)

### Formulation 5

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

### Example 3—Pellets

The drug substance powder can also be incorporated into a pellet that can produce a particle with improved flowability and density relative to the powder in Example 1, and better flowability than the granulations in Example 2. These pellets may be filled into a hard gelatin capsule and subsequently filled into bottles, or packaged in a sachet, stick pack, vial, or other container to aid in portability and ease of administration. Pellet formulations that may be used for topical application or dissolution into a topical solution may be prepared using similar formulations to those shown in Example 2, with the addition of 2 processing steps. This pellet formulation may be applied directly to the wound, or dissolved in an aqueous vehicle for topical administration. Pellet formulation examples are shown here:

### Formulation 6

Drug substance—700 g (active ingredient)
Microcrystalline cellulose—200 g (diluent and processing aid)
Povidone—100 g (binder)
Water (processing aid; removed during processing)

### Formulation 7

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing) Formulation 8
Drug substance—600 g (active ingredient)
Microcrystalline cellulose—300 g (diluent and processing aid)
Hydroxypropyl cellulose—100 g (binder)
Water (processing aid; removed during processing)

### Formulation 9

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)
   Step 1—add the active and excipients to a suitable pharmaceutical mixer or granulator such as a planetary mixer, high-shear granulator, fluid bed granulator, or extruder.
   Step 2—slowly add the water while the mixer is operating until all the water has been added.
   Step 3—continue the granulation step until the granulation endpoint is achieved.
   Step 4—Load the wet mass into a suitable extruder and extrude using a screen with apertures between 300 μm and 800 μm.
   Step 5—extrude the wet mass and introduce the extrudate into the marumerizer for pellet formation and spheronization.
   Step 6—discharge the wet mass of pellets into a container suitable to hold the material until drying.
   Step 7—charge the wet mass into a suitable drier and dry until the endpoint of less than 2% water is reached.
   Step 8—discharge the dried pellets into a suitable bin or container to store until the filling operation.
   Step 9—fill the appropriate amount of pellets into each package for storage, transfer, and administration.

### Example 4-Mini Tablets

The drug substance powder can also be incorporated into minitablets that can produce a particle with similar performance characteristics as a pellet. Minitablets offer another dry formulation approach, where a solvent may not be required. These minitablets typically have a diameter on the order of 500-2000 μm may be filled into a hard gelatin capsule and subsequently filled into bottles, or packaged in a sachet, stick pack, vial, or other container to aid in portability and ease of administration. This minitablet formulations may be applied directly to the wound, or dissolved in an aqueous vehicle for topical administration. Minitablet formulation examples are shown here:

### Formulation 10

Drug substance—600 g (active ingredient)
Microcrystalline cellulose—300 g (diluent)
Lactose—100 g (diluent)
Hypromellose (HPMC)—50 g (binder)
Colloidal silicon dioxide—50 g (glidant)
Magnesium stearate—5 g (lubricant)

### Formulation 11

Drug substance—600 g (active ingredient)
Silicified microcrystalline cellulose—400 g (diluent)
Hypromellose (HPMC)—50 g (binder)
Colloidal silicon dioxide—50 g (glidant)
Magnesium stearate—5 g (lubricant)

### Formulation 12

Drug substance—600 g (active ingredient)
Silicified microcrystalline cellulose—400 g (diluent)
Polyvinylpyrrolidone (PVP)—50 g (binder)
Colloidal silicon dioxide—50 g (glidant)
Magnesium stearate—5 g (lubricant)

### Formulation 13

Drug substance—700 g (active ingredient)
Silicified microcrystalline cellulose—250 g (diluent)
Polyethylene glycol (PEG)—50 g (binder)
Colloidal silicon dioxide—50 g (glidant)
Magnesium stearate—5 g (lubricant)

### Example 7—Cream

A topical cream formulation may be prepared using the following formula for the preparation of a batch of approximately 1 Kg:

### Formulation 14

Methyl paraben—0.25 g (preservative)
Propyl paraben—0.15 g (preservative)
Polysorbate 60—10 g (emulsifier)
Propylene glycol—120 g (viscosity modifier)
Stearyl alcohol—200 g (oleaginous phase)
White petrolatum—200 g (oleaginous phase)
Purified water—470 g (aqueous base)
   Step 1—mix the stearyl alcohol and white petrolatum and heat to approximately 75° C. to melt the base.
   Step 2—Dissolve the remaining excipients in the purified water by stirring until a solution is obtained.
   Step 3—Add approximately 5 g of drug substance to the purified water solution and mix for 5 additional minutes to dissolve the drug to manufacture a 5% ointment.
   Step 4—Slowly incorporate the aqueous solution to the oleaginous base and mix until it is well mixed.
   Step 5—Fill the cream into a suitable package such as a tube or pump bottle.

### Example 8—Gel

Topical gels can be used for sustained-release of actives, provide lubrication, and a carrier of pharmaceutical agents. Hydrogels are water-based and are less oily than creams or ointments, as well as exhibit excellent spreading properties, and may exhibit a higher retention time on the skin. Hydrogels can be simple formulations and may provide for a higher drug capacity than oil based formulations due to the high aqueous solubility of the drug substance. Gel formulation examples are shown here:

### Formulation 15

Drug Substance—50 g (active)
Carbopol—300 g (polymer/viscosity)
Purified Water—650 g (solvent)

### Formulation 16

Drug Substance—50 g (active)
Sodium carboxymethylcellulose—400 g (polymer/viscosity)
Purified Water—550 g (solvent)

### Formulation 17

Drug Substance—50 g (active)
Hypromellose—200 g (polymer/viscosity)
Purified Water—750 g (solvent)
   Step 1—Slowly add the polymer to the purified water while stirring slowly using a suitable mixer such as a Silverson mixer. Continue to mix until the polymer exhibits a lump-free dispersion.
   Step 2—Slowly add the drug substance to the polymer dispersion and mix until dissolved.
   Step 3—Fill the gel into a suitable package such as a tube or pump bottle.

### Example 9—Ointment

Hydrophilic ointment may be prepared using the following formula for the preparation of about 1 Kg of base:

### Formulation 18

Methyl paraben—0.25 g (preservative)
Propyl paraben—0.15 g (preservative)
Sodium lauryl sulfate—10 g (emulsifier)
Propylene glycol—120 g (viscosity modifier)
Stearyl alcohol—250 g (oleaginous phase)
White petrolatum—250 g (oleaginous phase)
Purified water—370 g (aqueous base)
   Step 1—mix the stearyl alcohol and white petrolatum and heat to approximately 75° C. to melt the base.
   Step 2—Dissolve the remaining excipients in the purified water by stirring until a solution is obtained.
   Step 3—Add approximately 1 g of drug substance to the purified water solution and mix for 5 additional minutes to dissolve the drug to manufacture a 1% ointment.
   Step 4—Slowly incorporate the aqueous solution to the oleaginous base and mix until it congeals.
   Step 5—Fill the ointment into a suitable package.

### Example 10—Sterile Solution for Subcutaneous or Intramuscular Administration

The drug may be incorporated into a solution for delivery via intramuscular (IM), subcutaneous (SC), or intravenous (IV) administration. Formulations designed to deliver active drug substances via the IM or SC route will generally have similar concentrations and volumes of administration. Formulations intended to provide 1 L of drug formulation for SC or IM administration are shown here:

### Formulation 19—10% (100 mg/mL)

Drug Substance—100 g (active)
Phosphate Buffer solution—1 L (solvent)
pH adjustment—0.1 N NaOH or 0.1 N HCl (pH adjustment)

### Formulation 20—5% (50 mg/mL)

Drug Substance—50 g (active)
Phosphate Buffer solution—1 L (solvent)
pH adjustment—0.1 N NaOH or 0.1 N HCl (pH adjustment)

### Formulation 21—1% (10 mg/mL)

Drug Substance—10 g (active)
Phosphate Buffer solution—1 L (solvent)
pH adjustment—0.1 N NaOH or 0.1 N HCl (pH adjustment)

### Formulation 22—0.1% (1 mg/mL)

Drug Substance—1 g (active)
Phosphate Buffer solution—1 L (solvent)
pH adjustment—0.1 N NaOH or 0.1 N HCl (pH adjustment)

### Formulation 23—0.05% (500 μg/mL)

Drug Substance—0.5 g (active)
Phosphate Buffer solution—1 L (solvent)
pH adjustment—0.1 N NaOH or 0.1 N HCl (pH adjustment)
   Step 1—Slowly add the drug substance to the buffer solution while stirring.
   Step 2—Continue stirring until a clear solution is obtained.
   Step 3—Measure the pH of the solution, and adjust to a pH of 6.5-7.5 using the dilute HCl or NaOH solution.
   Step 4—Sterile filtration using a 0.22 μm filter, and fill into a sterile syringe for a pre-filled syringe drug-device combination.

The above disclosure contains various examples of methods of treating Ebola virus disease and related diseases using peptoid compounds. Aspects of these various examples may all be combined with one another, even if not expressly combined in the present disclosure, unless they are clearly mutually exclusive.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

Furthermore, unless it is otherwise clear that a single entity is intended, terms such as “a,” “an,” and “the” are not intended to refer to only a singular entity and include the general class of which a specific example is described for illustration. In addition, unless it is clear that a precise value is intended, numbers recited herein should be interpreted to include variations above and below that number that may achieve substantially the same results as that number, or variations that are “about” the same number. Finally, a derivative as disclosed herein may include a chemically modified molecule that has an addition, removal, or substitution of a chemical moiety of the parent molecule.

Various compositions and instruments are identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

1. A method of treating or preventing an infection of a virus of the Filoviridae family in a subject, the method comprising:
administering to the subject an effective amount of one or more peptoid compounds selected from:
a peptoid compound H-Ndec-Nlys-Nspe-Nspe(p-Br)-Nlys-Npse-Nspe(p-Br)-NH₂,
a peptoid compound H-Ndec-(Nlys-Nspe-Nspe(p-Br))₂-Nlys-NH₂, and
a combination thereof,
thereby treating or preventing the infection.

2. The method of claim 1, wherein the virus is Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.

3. The method of claim 1, wherein the virus is Ebola virus.

4. The method of claim 1, wherein the administering is intranasal, oral, transdermal, transmucosal, intraperitoneal, subcutaneous, intramuscular, or intravenous, or any combinations thereof.

5. The method of claim 1, wherein the effective amount is from 1 mg/day to 1000 mg/day, or from 25 mg/day to 750 mg/day, or from 50 mg/day to 500 mg/day, or from 100 mg/day to 400 mg/day.

6. The method of claim 1, wherein the administering is once per day, twice per day, three times per day, four times per day, once per week, twice per week, once per two weeks, or once per month.

7. A method of inhibiting or preventing replication of a virus of the Filoviridae family, the method comprising:
contacting the virus with:
a peptoid compound H-Ndec-NLys-Nspe-Nspe(p-Br)-NLys-Npse-Nspe(p-Br)-NH₂,
a peptoid compound H-Ndec-(NLys-Nspe-Nspe(p-Br))₂-NLys-NH₂,
a peptoid compound H-NLys-NSpe(p-Br)-NSpe(p-Br)-NLys-NSpe(p-Br)₅-NSpe(p-Br)-NH₂,
a peptoid compound H-Ntetradec-NLys-Nspe-Nspe-NLys-NH₂,
or any combination thereof,
in an amount and for a time sufficient to inhibit or prevent replication of the virus.

8. The method of claim 7, wherein the virus is Lloviu virus, Měnglà virus, Bombali virus, Bundibugyo virus, Reston virus, Sudan virus, Taï Forest virus, Ebola virus, Marburg virus, Ravn virus, Xīlǎng virus, Huángjiāo virus, or any combination thereof.

9. The method of claim 7, wherein the virus is Ebola virus.

10. The method of claim 7, comprising:
binding the one or more peptoid compounds to a surface of an object, thereby conferring antiviral activity to the object.

11. The method of claim 7, comprising:
applying a composition comprising the peptoid to a surface of an object.

12. The method of claim 7, comprising:
applying a composition comprising the peptoid as a cleaning solution to a subject's skin.
